(19) United States
(12) Patent Application Publication
(10) Pub. No.: US 2011/0052545 A1
(43) Pub. Date: Mar. 3, 2011

(54) Regeneration system, its production and use

(76) Inventors: Katalin Vehmeyer, Gottingen (DE); Frauke Alves, Gottingen (DE)

(21) Appl. No.: 11/919,803

(22) PCT Filed: May 5, 2006
(86) PCT No.: PCT/EP2006/004239

§ 371 Date: Feb. 27, 2009

Related U.S. Application Data
(60) Provisional application No. 60/677,809, filed on May 5, 2005.

(51) Int. Cl. A61K 35/12; C12N 5/071; A61P 43/00; C12Q 1/02

(52) U.S. Cl. 424/93.7; 435/378; 435/29

(57) ABSTRACT

The present invention relates to a tissue-maintaining colony-forming unit (TM-CFU), a method of preparing the same, a pharmaceutical composition comprising the TM-CFU, the use of the TM-CFU for the manufacture of a pharmaceutical composition, and a method of determining the effect of at least one stimulus on the TM-CFU or a cellular subpopulation thereof.

The present invention relates to a tissue-maintaining colony-forming unit (TM-CFU), a method of preparing the same, a pharmaceutical composition comprising the TM-CFU, the use of the TM-CFU for the manufacture of a pharmaceutical composition, a method of treating a subject, and a method of determining the effect of at least one stimulus on the TM-CFU or a cellular subpopulation thereof.

Experimental biology and medicine work with stem cells has been performed for more than twenty years. The method discovered for in vitro culture of human embryonic stem cells acquired at abortions or from “surplus” embryos left from in vitro fertilization, evoked immediately ideas on the possibility to aim development and differentiation of these cells at regeneration of damaged tissues. Over 100 million Americans suffer from diseases that may eventually be treated more effectively with stem cells or even cured. However, the use of embryonic stem cells is limited due to legal barriers, ethical concerns and technical problems associated with embryonic stem cells.

Stem cells can also be extracted from adult tissue. There had been a consensus among researchers that adult stem cells were limited in usefulness as they were believed to produce only a few of the 220 types of cells in the human body. Recently, several surprising observations proved that even tissue-specific cells such as monocytes are capable, under suitable conditions, of producing a whole spectrum of cell types, regardless, whether these tissues are derived from the same germ layer or not. This ability is frequently called stem cell plasticity or “transdifferentiation”.

Therefore, somatic stem cells residing in various tissues are under intensive investigation for their possible use in cell therapy, especially in processes such as tissue repair. Particularly in the field of neurology, research has been intensified in order to establish new therapies for Parkinson's disease, Alzheimer's disease, multiple sclerosis and stroke. Furthermore, it is of particular interest to find cell therapies for diseases leading to the death of different cells. Examples of such diseases are diabetes mellitus, damages of the liver or of the myocardium or diseases of the kidney.

Somatic stem cells have been identified in almost all tissues. Apart from monocytes, their exact identity is still unknown. Monocytes of the peripheral blood have been shown to transdifferentiate into neural, epithelial cells, chondrocytes etc. under suitable conditions. However, the use of monocytes as stem cells is limited, since their differentiation into particular cells has to be stimulated using highly specific cytokines before transplanting the resulting cells and the knowledge of differentiation of many cell types such as neuronal cells or epithelial cells is still fragmentary.

Therefore, its one object of the present invention to provide an universal biological stem cell system for tissue repair which is capable of differentiating in vivo and in vitro in tissue-specific cell types without adding highly specific differentiation-inducing agents to the medium, whose use is not limited due to legal barriers and ethical concerns and which can be transplanted without inducing profound transplant reaction in the transplanted subject.

The object of the invention has been solved by a method of preparing a new type of colony derived from somatic cells, i.e. a tissue-maintaining colony-forming unit (TM-CFU). Surprisingly, the TM-CFU is capable of differentiating in vivo and in vitro into a series of tissue-specific cell types. The TM-CFU is the progeny of mononuclear phagocytic cells (MPCs). We proved that the mononuclear phagocytic system (MPS) develops from one cell. The TM-CFU is thus the in vitro equivalent of the physiological repair system. Surprisingly, it has been found that the cells of the TM-CFU can be used as in vitro cultivated biological repair system, e.g. for treatment of diseases and disorders associated with cell or tissue damage such as cancer, autoimmune diseases or neurodegenerative diseases. In this biological repair system cells such as dendritic cells (microglia, Langerhans cells, tissue macrophages, Kupfer cells etc.) are included. Monocytes are the mature representatives of MPCs in the blood. Under suitable culture conditions, e.g. as detailed below, mainly the immature dendritic cells grow showing differentiation potential among others for astrocytes, neurones, chondrocytes, epithelial cells and endothelial cells. However, a highly significant differentiation is the neuroectodermal differentiation.

Using the method of the present invention as detailed below, it is possible to cultivate a multipotent stem cell that is capable of developing large colonies which can give rise to both, the mesenchymal and the neuroectodermal differentiation. Surprisingly, it has been demonstrated that the method of the invention leads to the formation of a cellular colony consisting of stem cell-like cells, wherein the cells of the unity as a whole are capable of differentiating into a large number of different types of cells and/or tissues, preferably into any type of cell or tissue. The method of the invention does not involve the use of embryonic stem cells, but the use of adult stem cells. Accordingly, the method does not raise serious moral, ethical and religious concerns and/or legal problems associated with the use of embryonic stem cells. Additionally, the cells needed for the production of the TM-CFU of the invention are easily accessible. The TM-CFU of the present invention can be prepared and transplanted such that the risk of transplant rejection is minimized, e.g. either by using cells from umbilical blood or by administering a TM-CFU to a subject whose cells have been used for the preparation of this TM-CFU.

Accordingly, the object of the present invention is solved by a method of preparing a TM-CFU consisting of CD14 negative cells comprising the steps of:
(a) cultivating, in the presence of Granulocyte/Macrophage Colony-Stimulating Factor (GM-CSF) and/or Interleukin-3 (IL-3), cells from bone marrow, blood, umbilical cord or skin; and
(b) isolating said TM-CFU formed in step (a),
wherein the TM-CFU is further defined by the presence of
   (i) a majority of a first group of cells, wherein the cells are round with an eccentric nucleus and grow non-adherently, hillock-like in the center of the TM-CFU,
      a second group of cells, wherein the second group of cells includes cells with extensions and cells having cuboid- or triangle-shaped morphology and wherein the cells of the second group are adherent and larger than the cells of the first group and grow underneath the first group of cells,
      a third group of cells, wherein the third group of cells includes cells with extensions and spindle-shaped cells and wherein the cells are adherent, have variable morphology and grow around the second group of cells, and
      optionally satellite colonies developed in the center of the TM-CFU and showing embryoid body-like morphology;
   and/or
   (ii) the CD45 antigen.

The cells of the first, second and third group are also referred to as cells of type I, II or III or type I, II or III cells, respectively. A small number of cells of the first group may contain to more than one nucleus; these cells may be larger than the one with only one nucleus. On the surface of the colony there may be small satellite sometimes embryoid body-like colonies (clusters) consisting of small round cells capable of producing a new progeny. The cells of the third group may have one, two or more long extensions, wherein the extension can be as long as 20-30 times the diameter of the cell body. In a preferred embodiment of the invention about at most 25%, more preferably about 2 to 20%, still more preferably about 3 to 10% and most preferably about 5 to 7% of the cells of the second and third group have extensions.

In step a) of the method of the invention, cells from bone marrow, blood, umbilical cord or skin are used.

The cells from bone marrow can be obtained by e.g. bone marrow biopsy. If the sample is taken from a human, it is usually taken from the hip bone, but it can also be taken from other bones. The sample can be taken by cleansing the skin and injecting a local anesthetic to numb the skin. The biopsy needle can then be inserted into the bone. The core of the needle can then be removed, and the needle can be pressed forward and rotated in both directions. This forces a tiny sample of the bone marrow into the needle. The needle is then removed. Pressure can be applied to the biopsy site to stop bleeding and a bandage can be applied. The cells isolated in this manner can directly be used for step (a) of the method of the invention.

Preferably, the bone marrow sample is a liquid bone marrow blood sample obtained e.g. as detailed above. However, the sample could also be a non-liquid bone marrow sample.

Alternatively, cells from bone marrow can be obtained from e.g. the femur bone as detailed in the Examples.

The cells from blood can be obtained by taking a blood sample according to methods known to a skilled person such as a qualified doctor, nurse or anybody qualified in phlebotomy. In general taking a blood sample involves finding a blood vessel such as a vein or an artery and inserting a needle to extract the blood sample. In order to increase the number of stem cells in the blood sample it is possible to mobilize stem cells from the bone marrow to the bloodstream. For this, the donor can be treated with a hematopoietic growth factor, i.e. an agent causing blood cells to grow and mature, such as G-CSF, PEGylated G-CSF (Pegfilgrastim), GM-CSF, AMD3100 (AnorMed Inc., Canada) or G-CSF in combination with AMD3100 for e.g. 4 or 5 days before collecting of blood of the subject treated by using e.g. apheresis. The cells isolated in this manner can be directly used for step (a) of the method of the invention. In one embodiment of the invention erythrocytes and/or plasma is/are removed from the sample by e.g. centrifugation. The blood may be also overlayed onto Ficoll-Hypaque solution and centrifugated on the Ficoll-Hypaque solution to obtain mononuclear cells.

The amount of blood needed depends on the type of donor and its general condition such as state of health, age, sex, weight, fitness etc. However, the amount of apheresis product depends on the number of mobilized CD34⁺ cells. Preferably number of at least a 1×10⁷−CD34⁺ cells, typically a number of at least 5×10⁵-1×10⁶ CD34⁺ cells is necessary for the preparation of TM-CFU.

The cells for step (a) can be derived from umbilical cord. The blood of umbilical cords is rich in multipotent stem cell and umbilical cord blood stem cell allotransplants are usually less prone to rejection than either bone marrow or peripheral blood stem cells. Blood can be taken as described above for blood samples. Typically an amount of 10 to 20 ml of cord blood is sufficient for the preparation of TM-CFU.

Additionally, cells to be used in step (a) of the method of the invention can be derived from skin, e.g. obtained as follows: A piece of skin, e.g. approximately at least 0.5 cm² is taken from the donor, preferably under sterile conditions, disaggregated e.g. by mechanical, chemical and/or enzymatic treatment to obtain a cellular suspension (Kolcikova et al., 1998 J Immunol 1998, 161, 4033-4041). Thereafter, the suspension is cultivated e.g. overnight. Non-adherent cells can be decanted and cultivated further on as described below in order to obtain the TM-CFUs of the invention.

In a preferred embodiment of the invention the cells to be cultivated in step (a) have been obtained from a vertebrate, preferably a mammal such as a dog, cat, rabbit, rat, cattle, pig or sheep, more preferably a mouse or a human.

After having been obtained and optionally further purified e.g. as detailed above, the cells may be immediately cultivated or frozen for storage as known to the person skilled in the art. The cells may e.g. be frozen in the media as detailed above e.g. further comprising a cryoprotectant such as DMSO. The cells may be stored as detailed in the Examples.

The cells having been obtained from bone marrow, blood, umbilical cord or skin are cultivated in step (a) of the method of the invention, i.e. in the presence of GM-CSF and/or IL-3, preferably in the presence of GM-CSF, more preferably in the presence of GM-CSF and IL-3. The concentration of GM-CSF is preferably from 5 to 50 ng/ml, more preferably from 10 to 40 ng/ml, even more preferably from 20 to 30 ng/ml and most preferably approximately 25 ng/ml. The concentration of IL-3 is preferably from 5 to 50 ng/ml, more preferably from 10 to 40 ng/ml, even more preferably from 20 to 30 ng/ml and most preferably approximately 25 ng/ml. If cells are incubated with GM-CSF and IL-3, both substances may be added either consecutively or more preferably simultaneously.

Cells, either fresh or thawed, are cultivated in step (a) as known by the person skilled in the field of cell biology. The cells may be cultivated e.g. in a liquid medium or in a semi-liquid medium. For this, standard media can be used such as e.g. Dulbecco's Modified Eagle Medium (DMEM) or that described in the Examples such as Iscove's Modified Dulbecco's Medium (IMDM) optionally containing e.g. methylcellulose or agar for semi-solid cultures. Methylcellulose may be present e.g. from 0.5 to 3%, preferably from 0.7 to 2%, and more preferably at approximately 1% (vol/vol). Agar may be present in the medium e.g. from 0.03 to 3%, preferably from 0.1 to 1%, and more preferably at approximately 0.3% (vol/vol). The term “semi-solid culture” refers to cells cultivated in media with increased viscosity as compared to liquid media without converting the medium into a solid. Semi-solid media can be prepared by e.g. adding a gelling agent such as agar or methylcellulose to a liquid medium. This helps the clonal progeny of a single progenitor cell to stay together and facilitates the recognition and enumeration of distinct colonies.

Preferably the medium as detailed above is supplemented with serum, such as fetal calf serum (FCS). The concentration of the FCS may be at least 5%, preferably at least 10% and more preferably at least 20% of the medium (vol/vol). Most preferably, the medium is specified as detailed in the Examples. However, very preferred serums are “Fetal Bovine Serum for murine myeloid colony assay” (catalog #06200), “Fetal Bovine Serum for human myeloid colony assay” (catalog #06100), which are provided and pre-tested to ensure standardized serum conditions by StemCell Technologies.

Before starting cultivation, cells may be diluted to obtain a suitable concentration of cells in the medium. In a preferred embodiment the concentration can be e.g. from 1×10³ to 1×10⁵ cells/ml and most preferably from 5×10³ to 5×10⁴ cells/ml. Typically, a concentration of 5×10³ cells/ml and 1×10⁴-5×10⁴ cells/ml is used for murine and human donors, respectively. In another preferred embodiment, the cells are cultivated in a single cell format. Single cell format refers to a cultivation format, in which one single cell is present in one container at the beginning of the cultivation step (a) or in which the seeding density is so low that cells derived from different progenitors do not directly contact each other. For the single cell format, cells are preferably diluted to a larger extent. In a preferred embodiment the concentration can be e.g. from 10 to 1000 cells/ml, more preferably from 50 to 500 cells/ml and most preferably approximately 80 cells/ml. Accordingly, the volume of cell suspension added to one container is to be calculated based on the concentration of the cells in the suspension. If the concentration of cells amounts to approximately 80 cells/ml, preferably a volume of approximately 10 to 12 μl of the cell suspension should be added to each container. When cultivated in the single cell format, the TM-CFU of the invention is preferably derived from a single cell.

The total amount of cells cultivated in step (a) should be chosen in order to assure the presence of a stem cell capable of generating a TM-CFU according to the present invention. In bone marrow of healthy mice approximately 1 out of 1.000 cells is capable of generating a TM-CFU according to the present invention. If 10.000 cells isolated from bone marrow are cultivated, the probability of cultivating at least one cell capable of generating a TM-CFU is >99.9%. The density of cells capable of generating a TM-CFU in blood without mobilization of stem cells from bone marrow may amount to approximately 0.001%, in umbilical cord to approximately 0.05% and in bone marrow to 0.05-0.1%.

The person skilled in the art will recognize that the ratio of cells capable of generating a TM-CFU will depend on the type of donor and its general condition such as state of health, age, sex, weight, fitness etc. After chemotherapy the amount of stem cells in the blood normally increases due to mobilization of CD34⁺ cells. After repeated long lasting chemotherapy a significant reduction of progenitors is expected. Furthermore, diseases with repair requirements (e.g. rheumatisms, psoriasis) and/or with increased physiological cell turn over may dramatically increase the number of MPS progenitors in bone marrow, blood and tissues.

The cells may be cultivated at a temperature of from 25 to 40° C., preferably from 32 to 39° C., more preferably at 35 to 38° C. and most preferably at approximately 37° C. The cells can be grown in a humidified atmosphere of O₂ and CO₂, such as an atmosphere essentially consisting of from 90 to 98% O₂ and from 10 to 2% CO₂, preferably 92 to 95% O₂ and from 5 to 8% CO₂ and most preferably of approximately 93.5% O₂ and approximately 6.5% (vol/vol) CO₂.

In a preferred embodiment of the method of the invention Stem Cell Factor (SCF), Flt-3 ligand (FL) and/or Macrophage Colony Stimulatory Factor (M-CSF) is/are additionally present in step (a). The concentration of SCF is preferably from 1 to 100 ng/ml, more preferably from 10 to 30 ng/ml, even more preferably from 15 to 25 ng/ml and most preferably approximately 20 ng/ml. The concentration of FL is preferably from 1 to 50 ng/ml, more preferably from 2 to 30 ng/ml, even more preferably from 3 to 20 ng/ml and most preferably approximately 10 ng/ml. If M-CSF is used, the concentration of M-CSF is preferably from 1 to 100 ng/ml, more preferably from 10 to 50 ng/ml, even more preferably from 20 to 30 ng/ml and most preferably approximately 25 ng/ml. However, in one preferred embodiment the method of the invention is carried out in the absence of M-CSF.

In an even more preferred embodiment of the invention the following combinations of factors are present in step (a) of the method of the invention, preferably in the concentrations as detailed above:
GM-CSF, IL-3 and SCF;
GM-CSF, IL-3 and FL;
GM-CSF, IL-3 and M-CSF;
GM-CSF, IL-3, M-CSF and SCF;
IL-3 and M-CSF;
IL-3 and SCF;
IL-3, M-CSF and SCF;
GM-CSF and M-CSF;
GM-CSF and SCF;
GM-CSF, M-CSF and SCF;
FL and GM-CSF;
FL and IL-3;
FL, GM-CSF, IL-3 and M-CSF;
FL, GM-CSF, IL-3 and SCF;
FL, GM-CSF, IL-3, M-CSF and SCF;
FL, IL-3 and M-CSF;
FL, IL-3 and SCF;
FL, IL-3, M-CSF and SCF;
FL, GM-CSF and M-CSF;
FL, GM-CSF and SCF; or
FL, GM-CSF, M-CSF and SCF.

Additionally, TNF-α may be used in combination with the above factors, particularly in combination with IL-3, GM-CSF, M-CSF and SCF. The concentration of TNF-α is preferably from 1 to 100 ng/ml, more preferably from 5 to 50 ng/ml, even more preferably from 10 to 20 ng/ml and most preferably approximately 15 ng/ml.

In a still more preferred embodiment of the invention GM-CSF, IL-3 and SCF are present in step (a), most preferably during the complete incubation period. M-CSF may be additionally present, however, the combination of GM-CSF, IL-3 and SCF is the most preferred one. The concentration of GM-CSF, IL-3 and M-CSF (if present) is each from 1 to 100 ng/ml, more preferably from 5 to 50 ng/ml, even more preferably from 20 to 30 ng/ml and most preferably approximately 25 ng/ml, whereas the concentration SCF is preferably from 1 to 100 ng/ml, more preferably from 10 to 30 ng/ml, even more preferably from 15 to 25 ng/ml and most preferably approximately 20 ng/ml. Media of particular use in the method of the invention are exemplified in the Examples.

In another preferred embodiment of the method of the invention Leukemia Inhibitory Factor (LIF) is additionally present in step (a). The concentration of LIF is preferably from 100 to 10000 U/ml, more preferably from 200 to 5000 U/ml, even more preferably from 500 to 2000 U/ml and most preferably approximately 1000 U/ml.

In still another preferred embodiment of the invention the cultivation of step (a) is carried out over approximately 6 to approximately 16 days, preferably from approximately 7 to approximately 14 days, more preferably from approximately 8 to approximately 12 days, even more preferably from approximately 8 to approximately 10 days.

After the cells have been cultivated for a time sufficient to obtain a TM-CFU, the TM-CFU is isolated from the culture. The TM-CFU may be isolated as single cell clones e.g. from the methylcellulose cultures by using a pipette. If grown in the single cell format or freed from surrounding cells, the TM-CFU can also be isolated by cell scratcher and pipette, as well as from liquid culture.

Isolated clones may be further expanded in e.g. liquid cultures containing IMDM and 10% FCS (identical to methylcellulose culture conditions) and growth factors as indicated previously (GM-CSF, IL-3, SCF, M-CSF, FL and/or LIF, etc.). This is particularly useful, if larger amount of cells are needed for therapy or prophylaxis. In one embodiment of the invention only or mainly cells of the first group can be cultivated, since it has been shown that also cells of the second and third group develop from cells of the first group.

Another subject of the invention is a TM-CFU obtainable according to the method of the present invention, wherein the TM-CFU may be further characterized as detailed in the preferred embodiments of the TM-CFU or of the method of preparing the TM-CFU.

Still another subject of the invention is a TM-CFU consisting of CD14 negative cells, wherein the TM-CFU is further defined by the presence of
(i) a majority of a first group of cells, wherein the cells are round with an eccentric nucleus and grow non-adherently, hillock-like in the center of the TM-CFU,
   a second group of cells, wherein the second group of cells includes cells with extensions and cells having cuboid- or triangle-shaped morphology and wherein the cells of the second group are adherent and larger than the cells of the first group and grow underneath the first group of cells,
   a third group of cells, wherein the third group of cells includes cells with extensions and spindle-shaped cells and wherein the cells are adherent, have variable morphology and grow around the second group of cells, and optionally satellite colonies developed in the center of the TM-CFU and showing embryoid body-like morphology;
and/or
(i) the CD45 antigen.

The cells of the first, second and third group are also referred to as cells of type I, II or III or type I, II or III cells, respectively. A small number of cells of the first group may contain more than one nucleus; these cells may be larger than the one with only one nucleus. On the surface of the colony there may be small satellite sometimes embryoid body-like colonies (clusters) consisting of small round cells capable of producing a new progeny. The cells of the third group may have one, two or more long extensions, wherein the extension can be as long as 20-30 times the diameter of the cell body. In a preferred embodiment of the invention about at most 25%, more preferably about 2 to 20%, still more preferably about 3 to 10% and most preferably about 5 to 7% of the cells of the second and third group have extensions.

The TM-CFU of the invention is a novel high proliferative potential colony-forming unit (HPP-CFU), i.e. the progeny of a single cell giving rise in macroscopic colonies of at least 2-5 mm of diameter, preferably at least 3-5 mm of diameter, more preferably at least 4-5 mm of diameter.

The TM-CFU contains cells exhibiting mononuclear phagocyte- and neuron-like morphology. Surprisingly, it was found that 1) the mononuclear phagocytic system (MPS) develops from a common cell, 2) neural cell differentiation anchors within the MPS development, and 3) mesenchymal and neuroectodermal differentiation generates from one and the same progeny. It is noteworthy to highlight the presence of spindle shaped cells in the periphery of TM-CFUs, which express only weak CD45 protein and show mesenchymal stem cell morphology. The MPS comprises mobile cells which are constituents of tissue stroma and take part in the tissue homeostasis during physiological turnover, injury and developmental processes. In adult organisms, different members of the MPS show several common characteristics while differentiating to fulfill distinct organ specific functions. Mature monocytes showing a remarkable diversity generally leave the bone marrow and enter into the blood stream. From there, they migrate to various tissues where they transform into tissue macrophages. The cells of the MPS possess the remarkable ability to migrate to sites of tissue damage where they are involved in tissue regenerating processes. Additional representatives of the MPS are osteoclasts which play an essential role in bone remodeling, and dendritic cells such as Langerhans and microglial cells which are specialized antigen-presenting cells. The main function of dendritic cells is to initiate and regulate adaptive immune responses. The exact origin of different members of the MPS, exhibiting functional and phenotypic heterogeneity, has been unclear. However, using the method of the invention it is possible to produce a TM-CFU giving rise to all members of the MPS such as monocytes, macrophages, dendritic cells (e.g. Langerhans', microglial and Kupffer cells). However, the main population of TM-CFU represents an immature DC phenotype as described herein, e.g. characterized as follows: CD45⁺/CD11b⁺/CD11c⁺/HLA-DR⁻/CD14⁻. Since the cells of the novel high proliferative potential colony-forming unit (HPP-CFU) maintain tissue homeostasis, it is referred to as tissue-maintaining colony-forming unit (TM-CFU).

In one embodiment of the invention the TM-CFU is characterized by its typical appearance as detailed under item (i). A schematic representation is illustrated in FIG. 6 showing on top cells of the first group growing hillock-like and underneath adherent cells of the second group mainly as a monolayer of cells surrounded by the cells of the third group.

The TM-CFU of the present invention may be further characterized in that the cells of the first group are small cells. Additionally, the ratio of nucleus to cytoplasm of these cells is quite low, e.g. at least approximately 1:3, preferably at least approximately 1:4, more preferably from approximately 1:4 to approximately 1:5.

Depending on the conditions for cultivations, some of these cells may comprise more than one nucleus and accordingly be larger. The cells of the second group are larger. The diameter of the cells of the second group is from about 1.2 fold to about 2 fold, preferably about 1.5 fold, the diameter of the cells of the first group having one nucleus. The cells of the second group may be characterized by weak adherence and broad, elongated morphology. The cells of the third group may be characterized by strong adherence and diverse morphology. The third group includes gracile elongated spindle-shaped cells resembling cells of mesenchymal stem cell (MSC) origin, elongated cells with axon-like extensions, cells with dendrite-like extensions and/or cells having epithelial-like morphology. The spindle-shaped cells may grow radial around the second group of cells.

When cultivating cells according to step (a) of the method of the invention, colonies of cells may be obtained, which are not TM-CFU. This may be e.g. a colony resembling a large granulocyte-monocyte colony. This colony may be further characterized by the presence of few monocytic cells having large globular nuclei and several granulated cells and does not comprise a TM-CFU.

A further type of colony may be obtained characterized by a smaller diameter and irregular growth in the center of the colony, the cells growing in the center are larger than those of the first group of the TM-CFU and spindle-shaped cells are not present. This type of colony essentially consists of large and very large (up to 3-4 times larger as the first type cells of TM-CFU) round cells located in the middle of the colonies and very large adherent cells with extensions, which are usually not very long. Additionally, both colonies do not consist of the three groups of cells as detailed under item (i).

In a preferred embodiment of the invention the TM-CFU is further characterized in that the number of cells of the first group of cells amounts to at least 60%, preferably to at least 70%, more preferably to at least 80%, and even more preferably to at least 90% of the total number of cells of the TM-CFU.

In another preferred embodiment of the invention the TM-CFU is further characterized in that the number of cells of the second group of cells amounts to approximately 1% to 30%, preferably to approximately 2% to 20%, more preferably to approximately 3% to 10%, most preferably approximately 5% of the total number of cells of the TM-CFU.

In still another preferred embodiment of the invention the TM-CFU is further characterized in that the number of cells of the third group of cells amounts to approximately 1% to 30%, preferably to approximately 2% to 20%, more preferably to approximately 3% to 10%, most preferably approximately 5% of the total number of cells of the TM-CFU.

In a more preferred embodiment of the invention the number of the first group of cells amounts to approximately 80% to 94% of the total number of cells of the TM-CFU, the number of the second group of cells amounts to approximately 3% to 10% of the total number of cells of the TM-CFU and/or the number of the third group of cells amounts to approximately 3% to 10% of the total number of cells of the TM-CFU. Still more preferably, the number of the first group of cells amounts to approximately 90% of the total number of cells of the TM-CFU, the number of the second group of cells amounts to approximately 5% of the total number of cells of the TM-CFU and/or the number of the third group of cells amounts to approximately 5% of the total number of cells of the TM-CFU.

The TM-CFU may be further specified by its size. In another preferred embodiment of the invention the TM-CFU has a diameter of approximately at least 2 mm, preferably of approximately at least 3 mm, more preferably of approximately at least 4 mm, and most preferably of approximately at least 5 mm. More preferably, the diameter is measured after a cultivation period of from approximately 6 to approximately 16 days, preferably from approximately 7 to approximately 14 days, more preferably from approximately 8 to approximately 12 days, even more preferably from approximately 8 to approximately 10 days.

The TM-CFU may be further characterized by its number of cells. In another preferred embodiment of the invention the TM-CFU consists of at least 0.5×10⁴ cells, preferably at least 1.0×10⁴ cells, more preferably at least 1.5×10⁴ cells and most preferably at least 2.0×10⁴ cells. More preferably, the number of cells is determined after a cultivation period of from approximately 6 to approximately 16 days, preferably from approximately 7 to approximately 14 days, more preferably from approximately 8 to approximately 12 days, even more preferably from approximately 8 to approximately 10 days.

The cells forming the TM-CFU can be further characterized. According, in still another preferred embodiment of the invention the cells of the first group of cells have an average diameter of approximately 5 to 50 μm, preferably of approximately 10 to 40 μm, more preferably of approximately 15 to 30 μm, and most preferably of approximately 20 to 25 μm.

In another preferred embodiment of the invention the cell bodies of the second group of cells have an average diameter of at least approximately 25 μm, preferably of at least approximately 30 μm and most preferably of at least approximately 35 μm.

In a more preferred embodiment of the invention the cells of the first group of cells have an average diameter of approximately 15 to 30 μm and the cell bodies of the second group of cells have an average diameter of at least approximately 30 μm.

Apart from its appearance, the TM-CFU may be further specified by the presence and absence of particular markers. The presence and absence of these markers may be determined using methods known to the skilled person such as FACS analysis, immunostaining and/or cytochemical staining. The methods may be carried out e.g. as described in the Examples.

In one embodiment of the invention the TM-CFU of the invention consisting of CD14 negative cells is further characterized by the presence of the CD45 antigen (item (ii)). The term CD14 negative refers to the identification of CD14 antigen on the surface of cells using e.g. FACS analysis as detailed in the Examples. Preferably, the TM-CFU of the invention consists of CD14 negative cells in the context of the present invention, if less than 2% of the cells, more preferably if less than 1.5% of the cells, still more preferably if less than 1% of the cells, even more preferably if less than 0.1% of the cells, most preferably if none of the cells of the TM-CFU was identified as CD14 positive by the FACS. This may be determined e.g. using a FACScan cytometer and Quest software (Becton-Dickinson) (settings: Thres: 52, SSC: 300, FL2: 440) and may be carried out as detailed in the Examples.

As detailed above, the TM-CFU is characterized by the presence of the CD45 antigen. In a preferred embodiment of the invention the TM-CFU is characterized by the presence of the CD45 antigen. In a preferred embodiment of the invention at least 80%, preferably at least 85%, more preferably at least 90% and most preferably at least 95% of the total number of cells of the TM-CFU are CD45 positive. The presence of the CD45 antigen on the surface of the cells can be tested using e.g. FACS analysis e.g. as detailed in the Examples. In a particular embodiment of the invention the cells of the first group are very strong CD45 positive, the cells of the second group are strong CD45 positive and the cells of the third group show either significantly diminished or no CD45 expression at all.

In another preferred embodiment of the invention the cells of the TM-CFU are alkaline phosphatase (AP) negative or HLA-DR II negative. More preferably, the cells are AP negative and HLA-DR II negative. The term “negative” with respect to a particular marker refers to a signal which is not significantly different from the background signal; in a preferred embodiment the term can also be interpreted according to the definition for the CD14 marker. The presence and absence of these marker may be determined using e.g. FACS analysis, immunostaining, cytochemical staining and/or PCR e.g. as described in the Examples. Preferably, the cells of the TM-CFU of the invention are negative for a particular marker, e.g. HLA-DR, if less than 1% of the cells, more preferably if less than 0.1% of the cells, most preferably if none of the cells of the TM-CFU was identified as positive for that marker. “Positive for a marker” refers to a signal obtained in the respective analytical method, which is significantly different from that of the control value representing a negative control such as the background level. To quantify the expression of a particular marker, the number of cells positive may be counted or estimated.

In another preferred embodiment of the invention the TM-CFU is further characterized by the presence of at least one of the following markers:
F4/80 (indicative of macrophages);
CD11c (indicative of dendritic cells);
Glial Fibrillary Acidic Protein (GFAP; indicative of glia cells); and/or
Neuronal Nuclei (NeuN; indicative of neurons).

More preferably, the TM-CFU of the invention is characterized by the presence of the following combinations of markers:
CD45 and F4/80;
CD45 and CD11c;
CD45 and GFAP;
CD45 and NeuN;
F4/80 and CD11c;
F4/80 and GFAP;
F4/80 and NeuN;
CD11c and GFAP;
CD11c and NeuN;
GFAP and NeuN;
CD45, F4/80 and CD11c
CD45, F4/80 and GFAP
CD45, F4/80 and NeuN;
CD45, CD11c and GFAP;
CD45, CD11c and NeuN;
CD45, GFAP and NeuN
F4/80, CD11c and GFAP;
F4/80, CD11c and NeuN;
F4/80, GFAP and NeuN
CD11c, GFAP and NeuN;
F4/80, CD11c, GFAP and NeuN;
CD45, CD11c, GFAP and NeuN;
CD45, F4/80, GFAP and NeuN;
CD45, F4/80, CD11c, and NeuN;
CD45, F4/80, CD11c and GFAP; or
CD45, F4/80, CD11c, GFAP and NeuN.

In a more preferred embodiment of the invention the TM-CFU is characterized by the presence of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 36, 37, 38, 39, 40, 41, 42, 43, 44 or 45 of the markers selected from the group consisting of CD11b, CD90, CD91, S-100, CD205, CD115, CD117, MAC3, CD163, F4/80, CD86, CD80, CD34, CD31, pan cytokeratin, CD11c, CD135, vimentin, nestin, GFAP, Ibal, synapthophysin, NeuN, MAP-2a,b, class III β-tubulin, NSE, NF-200, E-cadherin, albumin, alpha-fetoprotein, TIMP-2, MMP-9, MMP-2, MMP-3, MMP-1, BMP4, BMP5, laminin, fibronectin, collagen type IV, collagen type II, actin, monocyte-specific esterase (MSE), tartrate resistant acid phosphatase (TRAP) and VIP (vasoactive intestinal peptide). If one of the markers shows a catalytic activity, its presence may be determined by measuring its catalytic activity. Catalytic activity can be determined as known by the skilled person or e.g. as detailed in the Examples.

In an even more preferred embodiment of the invention the TM-CFU is characterized by the presence of at least 1, 2, 3, 4, 5 or more markers indicating the presence of
at least one progenitor of monocytes, macrophages, dendritic cells and/or osteoclasts such as CD11b, CD11c, (CD80), (CD86), F4/80, CD163, MAC3, CD115, CD205, S-100, or CD91, CD90, CD34, CD117;
at least one progenitor of neuronal cells including glia cells such as nestin, NSE, NF-200, class III β-tubulin, microtubule-associated protein-2a,b (MAP-2a,b), NeuN, synapthophysin, Ibal or GFAP; and/or
at least one morphogenesis-associated protein such as E-cadherin, actin, collagen type II, collagen type IV, fibronectin, vimentin, laminin, MMP-1, MMP-3, pan cytokeratin, albumin, alpha-fetoprotein, BMP4, BMP5, MMP-9, MMP-2 or TIMP-2.

In another even more preferred embodiment of the invention the TM-CFU is characterized by the presence of at least 1, 2, 3, 4, 5 or more markers indicating the presence of
epithelial differentiation such as E-cadherin and/or pan cytokeratin;
endothelial differentiation such as CD31;
endodermal differentiation such as alpha-fetoprotein;
(primitive) neuroectoderm formation such as vimentin;
neuronal differentiation such as nestin, NSE, NF-200, class III β-tubulin, MAP-2a,b, NeuN, synapthophysin and/or GFAP; and/or
chondrocyte differentiation such as collagen type II and/or IV.

In the context of the present invention, a particular marker is present, if the actual value obtained in the detection method and determined for the TM-CFU is significantly different from the reference value. The reference value may e.g. be the background level of the detection method used. Preferably, the marker is present on the surface of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58 or 59%, more preferably at least 60%, even more preferably at least 80% of the cells tested.

Typical values for the expression determined by immunostaining of the markers are: (very strong expression: +++++, strong expression. ++++, middle expression. +++, weak expression: ++, very weak expression: +, each referring to ratio of positive cells)

| | |
|---|---|
| CD45 | +++++ |
| CD14 | no expression |
| MHC class II | no expression |
| CD90 | +++(+) |
| F4/80 | +++ |
| CD205 | +++ |
| MAC-3 | +++ |
| CD91 | +++ |
| CD163 | +++ |
| Iba-1 | ++++ |
| CD115 | ++ |
| CD34 | ++ |
| CD117 | ++(+) |
| CD135 | ++ |
| Vimentin | ++ |
| E-cadherin | ++++ |
| Fibronectin | +++++ |
| Collagen type II | +++ |
| Laminin | +++++ |
| MMP-1 | + |
| MMP-3 | + |
| TIMP-2 | + |
| S100 | ++++ |
| Nestin | +++++ |
| NSE | ++++ |
| GFAP | +++ |
| NeuN | ++(+) |
| Class III^{β}-tubulin | ++ |
| Synaptophysin38 | +++ |
| NF-200 | +++ |
| MAP-2a,b | +++ |
| CD 31 | ++ |
| Pan Cytokeratin | ++(+) |
| Albumin | + |
| Alpha Fetoprotein | + |
| Actin | +(+) |
| BMP 4 | + |
| BMP 5 | + |

Typical values for the expression determined by FACS analysis of the markers are: (The numbers define the ratio of positive cells)

| | |
|---|---|
| CD45-FITC (30-F11) | >90% |
| CD11b-PE (M1/70) | >80% |
| CD11c-PE (HL3) | >80% |
| CD80 | 10% |
| CD86 | 10% |

Typical values for the expression determined by PCR of the markers are: (very strong expression: +++++, strong expression: ++++, middle expression. +++, weak expression: ++, very weak expression: +)

| | |
|---|---|
| MMP-9 | +++++ |
| MMP-2 | +++ |
| MMP-3 | + |

Typical values for the activity of enzymes determined by cytochemical staining: (very strong activity. +++++, strong activity: ++++, middle expression: +++, weak expression: ++, very weak expression: +)

| | |
|---|---|
| MSE | +++++ |
| TRAP | +++++ |
| AP | no activity |

Typical values for the presence of hormones determined by cytochemical staining: (very strong activity: +++++, strong activity. ++++, middle expression: +++, weak expression: ++, very weak expression: +)

| | |
|---|---|
| Glucagon | (+) |
| Insulin | (+) |
| Vasoactive intestinal peptide | ++ |

In another preferred embodiment of the invention the cells of the TM-CFU show phagocytic activity. Phagocytic activity can be tested e.g. as detailed in the Examples.

In still another preferred embodiment of the invention the cells of the TM-CFU are capable of spontaneously differentiating into cells of the mononuclear phagocytic system and/or neural cells without adding a differentiation-inducing agent, in particular an agent inducing neuronal differentiation, to the medium used for cultivation.

In yet another preferred embodiment of the invention the TM-CFU is not derived from a differentiated cell, particularly not from a monocyte.

In another preferred embodiment of the invention the TM-CFU is derived from a vertebrate, more preferably a mammal such as a dog, cat, rabbit, rat, cattle, pig or sheep, even more preferably a mouse or a human.

The TM-CFU may be further characterized by one ore more features as detailed above with respect to the preferred embodiments of the method of preparing a TM-CFU.

Another subject of the invention is a pharmaceutical composition comprising the TM-CFU according to the present invention and optionally excipients and/or auxiliaries.

As a rule, it will be irrelevant for clinical use, if some of the cells present in the pharmaceutical preparation do not fulfill the criteria of the TM-CFU or if the TM-CFU is incomplete. However, the incomplete TM-CFU should contain at least 10%, more preferably at least 25%, still more preferably at least 50% and most preferably at least 75, 80, 90, or 95% of the cells of the complete TM-CFU. Particularly, a sufficient amount of each group of cells should be present in the pharmaceutical composition. It is also possible to combine the cells of two or more TM-CFUs into one pharmaceutical composition.

In a preferred embodiment the number of TM-CFU to be administered to a subject amounts to at least 10, more preferable at least 20, still more preferably at least 50 TM-CFU per treatment. It might be necessary to administer the TM-CFUs in several doses, e.g. on different days for successful treatment.

In another preferred embodiment the cells of the TM-CFU are propagated before administration. For this, TM-CFU are disaggregated into single clones or cell clusters which may be further expanded in e.g. liquid cultures containing IMDM and 10% FCS (identical to methylcellulose culture conditions) and growth factors as indicated previously (GM-CSF, IL-3, SCF, M-CSF, FL and/or LIF, etc.). This is particularly useful, if larger amount of cells are needed for therapy or prophylaxis. In one embodiment of the invention only or mainly cells of the first group can be cultivated, since it has been shown that cells of the first group were able to generate cells of the second and third group. Accordingly, it is sufficient to isolate cells of the first group for propagation. Additionally, it was found that it is not necessary to administer the complete TM-CFU, as detailed above.

For administration the TM-CFU of the propagated cells should be in a pharmaceutical dosage form in general consisting of a mixture of ingredients known to a skilled person in to the pharmacotechnical arts such as pharmaceutically acceptable excipients and/or auxiliaries combined to provide desirable characteristics. Examples of such substances are isotonic saline, Ringer's solution, buffers, organic or inorganic acids and bases as well as their salts and buffer solutions, sodium chloride, sodium hydrogencarbonate, sodium citrate or dicalcium phosphate, glycols, such a propylene glycol, sugars such as glucose, sucrose and lactose, starches such as corn starch and potato starch, albumins, organic solvents, complexing agents such as citrates and urea, stabilizers, such as protease or nuclease inhibitors, The physiological buffer solution preferably has a pH of approx. 6.0-8.0, especially a pH of approx. 6.8-7.8, in particular a pH of approx. 7.4, and/or an osmolarity of approx. 200-400 milliosmol/liter, preferably of approx. 290-310 milliosmol/liter. The pH of the pharmaceutical composition is in general adjusted using a suitable organic or inorganic buffer, such as, for example, preferably using a phosphate buffer, tris buffer (tris(hydroxyl-methyl)ami-nomethane). In general, the cells of the TM-CFU should be formulated and stored, e.g. by freezing, in order to facilitate viability of the cells by choosing appropriate conditions as known to the skilled person.

The pharmaceutical composition of the present invention can be administered to a subject by any route suitable for the administration of viable cells. Examples of such routes are intravascularly, intracranially, intracerebrally, intramuscularly, intradermally, intravenously, intraocularly, intraperitoneally, orthotopically in an injured organ or by open surgical procedure. The pharmaceutical composition may be administered to the subject by e.g. injection, infusion or implantation. It may be administered orthotopically, directly to the tissue or organ to be treated or reconstituted, i.e. the target tissue or target organ, or to a distant site. In one embodiment of the invention the pharmaceutical composition is injected into the peritoneum. Most preferably, the pharmaceutical composition is administered intravenously, intraperitoneally or orthotopically in an injured organ or by open surgical procedure.

Another subject of the invention relates to the use of a TM-CFU according to present invention for the manufacture of a pharmaceutical composition for the generation of a target cell or a tissue in a subject and/or for the regeneration of a tissue in a subject. The target cell or tissue may be any cell or tissue. Preferably the cell or tissue is located in the body of a subject to be treated. If the pharmaceutical composition containing the TM-CFU is administered to the subject, the cells of the TM-CFU will be applied to or migrate mainly to a target tissue or organ, e.g. an injured tissue or organ. In the environment of this tissue or organ, new cells forming or regenerating the organ or tissue will be generated by differentiating the cells of the TM-CFU into the respective cells.

Still another subject of the invention relates to a method of treating a subject being in need of maintaining, generating or regenerating a tissue, comprising administering to the subject an effective amount of cells from the TM-CFU in the present invention. As detailed above, it might be sufficient to administer an incomplete TM-CFU to a subject. Under other circumstances it might be necessarily to administer a combination of TM-CFU, in particular in connection with subjects suffering from e.g. severe organ or tissue damages. The specific therapeutically effective amount of cells for any particular subject will depend upon a variety of factors including the condition or disease the subject is suffering from, the route of administration, the age, body weight and sex of the patient, the duration of the treatment and like factors well known in the medical arts.

In a preferred embodiment of the invention tissue to be (re)generated is an endodermic, mesodermic and/or an ectodermic tissue.

In more preferred embodiment of the invention the tissue is located in an organ selected from the group consisting of the skin, the eye, the nose, the ear, the brain, the spinal cord, a nerve, the trachea, the lungs, the mouth, the esophagus, the stomach, the liver, the small intestines, the large intestines, the kidney, the ureter, the bladder, the urethra, a gland such as hypothalamus, pituitary, thyroid, pancreas and adrenal glands, the ovary, the oviduct, the uterus, the vagina, a mammary gland, the testes, the penis, a lymph nodes, a vessel, the heart, a blood vessel, a skeletal muscle, a smooth muscle, a bone, cartilage, a tendon and a ligament.

The TM-CFU may be used to prepare a pharmaceutical composition to be administered to a subject suffering a pathological condition or a disease. A pathological condition is any abnormal condition of the body of the subject.

In a preferred embodiment the pathological condition or disease is selected from the group consisting of from cancer, an autoimmune disease, a neurodegenerative disease, a respiratory disease, a vascular disease, diabetes mellitus, Alzheimer's disease, Lewy body dementia, Parkinson's disease, a trauma, burn, head trauma, spinal cord injury, stroke, myocardial infarction, arthrosis, Huntington's disease, Tourette's syndrome, multiple sclerosis, amyotrophic lateral sclerosis, Addison's disease, pituitary insufficiency, liver failure, inflammatory arthropathy, neuropathic pain, blindness, hearing loss, arthritis, a bacterial infection, a viral infection, a sexually transmitted disease and a damage of the skin, the eye, the nose, the ear, the brain, the spinal cord a nerve, the trachea, the lungs, the mouth, the esophagus, the stomach, the liver, the small intestines, the large intestines, the kidney, the ureter, the bladder, the urethra, a gland such as hypothalamus, pituitary, thyroid, pancreas and adrenal glands, the ovary, the oviduct, the uterus, the vagina, a mammary gland, the testes, the penis, a lymph nodes, a vessel, the heart, a blood vessel, a skeletal muscle, a smooth muscle, a bone, cartilage, a tendon or a ligament.

Additionally, the TM-CFU of the invention may e.g. be used
to investigate the biological role of MPS e.g. under physiological and pathological conditions, especially in disease with extremely activated MPS (respiratory disease, multiple sclerosis, rheumatism, psoriasis, etc . . . );
to study the biology (e.g. the phenotypic composition) of TM-CFU;
to study the release of biological active substances such as cytokines, enzymes, hormones, MMPs, adhesion molecules (e.g. in vitro, under physiological and/or pathological conditions and/or under the influence of biological active compounds e.g. cytokines, peptide hormones, synthetic peptides);
to study the role (significance) of MPS in the neuro-endocrine-immune-system;
to study the regulation of MPS by a compound such as cytokines, peptide hormones, synthetic peptides etc.;
to screen the effects of of compounds such as cytokines, peptide hormones, synthetic peptides etc. on MPS.
to investigate the participation of MPS in regeneration in vitro and in vivo;
to study in vitro the metamorphoses of MPCs using distinct differentiation stimulating protocols;
to studying the regeneration of injured tissue by transplanted MPCs in vivo;
to study the metamorphosis of MPCs into specific tissue-related cells; and/or
to test the influence and possible use of pharmaceutically active substances as for example pro-inflammatory mediators, cell adhesion- and migration-related substances, hormones, peptide hormones, synthetic peptides etc. on different MPS-related functions (importantly in inflammatory and regenerative processes).

The important scientific findings of investigations mentioned above should help to work out biological strategies in distinct diseases, moreover, to learn more about agents, drugs and/or pharmaceuticals for the prevention and treatment of diseases including the endogenous replenishment of e.g. injured tissue cells instead of exogenous stem cell transplantation.

Accordingly, another subject of the invention relates to a method of determining the effect of at least one stimulus on the TM-CFU according to any of claims 24 to 27 or a cellular subpopulation thereof:
(a) exposing the TMCFU to the at least one stimulus; and
(b) determining the effect of the at least one stimulus on the TM-CFU or a cellular subpopulation thereof.

The stimulus can be any stimulus such as a physical or chemical stimulus. Examples of a physical stimulus are heat, cold, electricity, radiation, light etc. Examples of a chemical stimulus are natural occurring, semi-synthetic or synthetic compounds or mixtures thereof. They may be part of a compound library in order to identify a compound having a particular effect on the TM-CFU or part thereof. For the method of the invention, the TM-CFU or part thereof is exposed to the stimulus under appropriate conditions and for an appropriate time. The skilled person will be able to determine the suitable conditions for the exposure. Simultaneous or after that, the effect of the exposure is determined. The effect may be any change induced by the stimulus in comparison to a TM-CFU which has not been subject to the exposure. This might be altered proliferation, differentiation, morphology, viability etc. of the TM-CFU or part thereof, such as a subpopulation of cells.

The method may be used in order to screen for a stimulus such as a compound which induces, promotes, enhances, inhibits, diminishes or reduces differentiation into a to particular cell type. Such a compound may be used to stimulate or inhibit production of a particular cell type in vivo or in vitro.

The following Examples and Figures are intended to illustrate the present invention, but not to limit the scope of the claims.

### FIGURES

FIG. 1. Morphology of TM-CFU (Tissue-Maintaining Colony Forming Unit) and TM-CFCs (Tissue-Maintaining Colony Forming Cells). (A) The most common morphology of a TM-CFU propagated in methylcellulose culture; (B) other possible forms of a TM-CFU in which the heap-like growing cells in the center of TM-CFU breaks of into clusters. Light microscopic images show (C) MSE-staining of TM-CFU-composing adherent cells in-situ grown in the presence of EGF and GM-CSF, (D) cytocentrifuge preparation of an isolated TM-CFCs after hematoxylin staining, (E) the heterogenous morphology of the TM-CFU composing adherent cells, and (F) F4/80-expressing multinucleated osteoclast-like giant cell.

FIG. 2. (A) Cross-sections of TM-CFU showing the three main parts of the colony: I. heap-like growing round cells; II. adherent cells underneath of the center of colony; III. surrounding adherent cells. Light microscopic images demonstrate (B) the surrounding adherent cells of TM-CFU showing mesenchymal cell-like morphology; (C) dendritic cell-like morphology in a TM-CFU, and (D, E, F) the heterogenous morphology of the TM-CFCs.

FIG. 3. Hemopoietic origin of TM-CFCs and evidence for the presence of a macrophage/antigen-presenting cell population. Flow cytometric analyses of CFCs with antibodies to (A) CD45 demonstrate hemopoietic origin of CFCs and to (B) CD11b, and (C) CD11c indicate the presence of a macrophage/antigen-presenting cell population. (D) Compatible with an immature cell population, no MHC class II antigen expression could be detected. Data are representative of 2-4 independent experiments. Dotted lines of histograms represent expression levels of the markers indicated, continuous lines of histograms represent controls.

FIG. 4. (A-F) Morphology of different colonies which may be associated with the TM-CFU growth in the methylcellulose cultures.

FIG. 5. Phagocytic activity of CFCs. (A, B) Phagocytosis of FITC-labelled beads by TM-CFCs. Light microscopic images show (C) CFCs prior to phagocytosis, and (D) phagocytosis of necrotic liver tissue by CFCs in a 10 days old co-culture. Note, the morphological changes of CFCs in (D) compared to (C): elongated cells became more and more round and show darkly stained cytoplasm.

FIG. 6. Schematic representation of the TM-CFU showing the type I, II and III cells in a lateral view (A), top view (B) and from a cut (C). Type II cells are below hillock-like type I cells and are surrounded by type III cells. The drawings are not exactly proportional to reality.

FIG. 7. (A) Human TM-CFU of the bone marrow from a patient growing in agar culture. Note the halo-like growing type III adherent cells. (B) Cytocentrifuge preparation of this colony after Giemsa-staining.

FIG. 8. Small embryoid body-like satellite colony. Arrow indicates satellite colony with embryoid body-like morphology growing in methylcellulose culture supplemented with GM-CSF, IL-3, M-CSF, SCF and LIF.

FIG. 9. In vivo detection of TM-CFCs. (A) GFP-expressing cells in bone marrow. Isolated cells from the bone marrow, cytospin, light microscopy (left), fluorescence microscopy (middle) and overlay of both (right). (B) GFP-expressing cells in spleen. Isolated cells from the spleen, cytospin, light microscopy (left), fluorescence microscopy (middle) and overlay of both (right). (C) GFP-expressing cells in the pancreas. Pancreas cryosection HE staining (left) and evidence of green GFP expressing cells (middle). Cellular evidence of green GFP expressing cells overlaid to Dapi nucleus staining in the pancreas (right).

### EXAMPLES

1. Isolation and Cultivation of Cells from Bone Marrow

Cells from bone marrow were obtained from 10-12 weeks old mice of both sexes of two inbred strains C57BL/6J (Charles River) and BALB/c (Winkelbach). Mice were killed by cervical dislocation and femur bones were instantly removed under sterile conditions. Mononuclear cells (MNCs) from femur were obtained by flushing out cells by a 23-gauge syringe using one mL Iscove's modified Dulbecco's Medium (IMDM, Seromed). Cells were cultivated either directly or alternatively after being frozen in IMDM supplemented with 20% fetal calf serum (FCS, Gibco BRL) and 10% dimethyl sulfoxid (Sigma). In total, about 600 independent cultures from 20 independent experiments were performed.

Semi-Solid Cultures:

Agar cultures were prepared according to the method described by Pragnell et al. (1988, Blood 72, 196-201). Briefly, MNCs (1×10⁴ cells per mL) were suspended in 0.3% agar medium and plated over 0.5% agar containing growth factors: Interleukin-3 (IL-3), GM-CSF and macrophage-colony stimulating factor (M-CSF) each at a concentration of 25 ng/mL, and 20 ng/mL stem cell factor (SCF; R&D Systems). Cultures were incubated up to three weeks at 37° C. under a fully humidified atmosphere and 6.5% CO₂. Colonies (>5 mm) were scored by using a dissection microscope (Zeiss Inc.):

Before use methylcellulose was pretested in order to assure comparable homogeneous TM-CFUs growth. Methylcellulose/serum was accepted if resulting TM-CFUs showed characteristic appearance as detailed above. Methylcellulose cultures were performed by transferring unselected MNCs of bone marrow (0.5×10⁴ cells per mL) or Lin⁻/Sca-1⁺/c-kit⁺ cells of bone marrow (1×10³ cells per ml) to 1% pretested methylcellulose medium containing 20% FCS (CellSystems) and supplemented with growth factors: Interleukin-3 (IL-3), GM-CSF and macrophage-colony stimulating factor (M-CSF) each at a concentration of 25 ng/mL, 20 ng/mL stem cell factor (SCF) and 10 ng/mL FL, ligand of FMS-like tyrosine kinase 3 receptor (R&D Systems). Some cultures were additionally supplemented with 1000 U/mL leucocyte inhibitory factor (LIF, R&D Systems) or 50 ng/mL epidermal growth factor (EGF, Gibco BRL) or 50 ng/mL nerve growth factor (NGF, Promega). To stimulate major histocompatibility complex (MHC) molecule class III-Ab antigen expression 100 U/mL mouse-interferon-□ (IN\tilde{F}□Roche) were added to cultures daily for three days.

Murine lineage-negative hematopoietic progenitor cells (Lin⁻/Sca-1⁺/c-kit⁺) cells were isolated and enriched with EasySep PE selection cocktail and EasySep magnetic nanoparticles (CellSystems) according to the method of the manufacture. The used method is designed for positive selection.

Cells were cultivated in either 8-well-chamber slide plates (0.25 mL per well; Falcon) or 2-well Lab-Tek chamber slides (0.5 mL per well; Nunc) at 37° C. under a fully humidified atmosphere and 6.5% CO₂. Colonies (>5 mm) were observed and scored for a period of 10-14 days using an inverted microscope (Zeiss Inc.).

Cultivation of cells in a single cell assay format: To ensure that colonies originated from single cells or grow TM-CFUs in a single cell format, MNCs were diluted in a mixture of methylcellulose and 20% FCS-containing IMDM (1:1) supplemented with IL-3, GM-CSF, M-CSF, and SCF as described above. Wells of flat-bottom HLA plates (Nunc) were filled with 0.01 mL cell suspension (about 80 cells per mL). Microscopical observation demonstrated the existence of single cells in approximately 30% of wells. At day nine, colonies which developed from single cells were transferred in 20% FCS-containing IMDM supplemented with growth factors as described above. After two days of incubation in 2-well chamber slides the culture medium was carefully removed, cells were then fixed with acetone/methanol (1:1) and immunostaining was performed using antibodies to nestin, glial fibrillary acidic protein (GFAP) and F4/80.

Expansion of the TM-CFC in liquid cultures: TM-CFUs were transferred in liquid cultures containing IMDM, 10% FCS (identical with methylcellulose) and GM-CSF and Interleukin 3 (25 ng/ml). Every three days half of the medium was replaced.

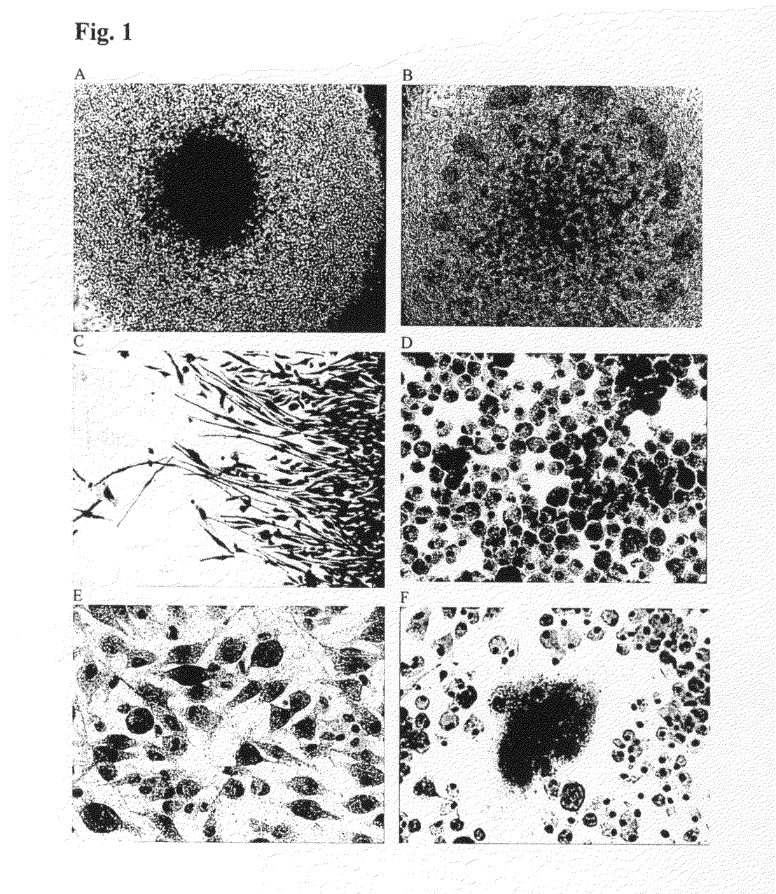

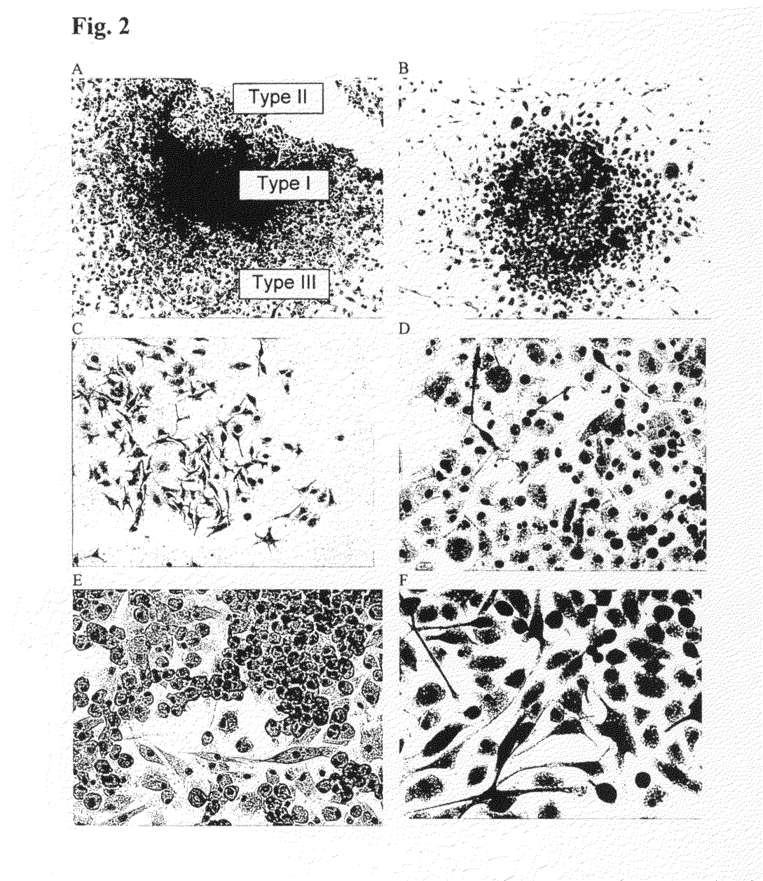

Results To investigate high proliferative potential colony-forming cells (HPP-CFCs), bone marrow cells isolated from femoral bones of adult mice were propagated in either agar or methylcellulose cultures in the presence of growth factors as indicated. Under these culture conditions, the development of large colonies of approximately 1-2×10⁴ cells was observed within 8-10 days (FIG. 1 and FIG. 2). Different types of colonies could be distinguished. The colony which was ultimately shown to be TM-CFU contained many tightly packed large and small round cells growing hillock-like in the center of colonies within methylcellulose (FIG. 1A). In some colonies several of the small round cells were clustered into small aggregates (FIG. 1B) surrounding adherent cells within the TM-CFU characteristically. Adherent cells within this type of colony characteristically exhibited a gracile elongated morphology with long and short extensions (FIG. 1C). FIG. 1D shows cells of an isolated TM-CFU on a cytocentrifuge preparation. The frequency of TM-CFUs was 3-7 per 5×10³ cells, although a 2-7-fold enrichment was observed when murine lineage-negative hematopoietic progenitor cells (Lin⁻/Sca-1⁺/c-kit⁺; 85% purity) were used. Similar results were obtained from bone marrow samples of mice strains C57BL/6J (n=15) and BALB/c (n=3). Cells that were recovered from liquid nitrogen storage developed some less colonies (2-5 TM-CFUs per 5×10³ cells). In order to be able to perform a detailed analysis of colony-forming cells we performed our investigations using 2-8-well chamber slide plates allowing morphological studies as well as expression analyses of different antigens in-situ.

The advantage of the methylcellulose culture system is the possibility to perform morphological studies as well as expression analyses of different antigens in-situ. Adherent cells in methylcellulose cultures displayed numerous long or short, partly ramified extensions that reached occasionally a length of up to 8-10 times the size of the respective cell bodies. Some of these cells were of large triangle shaped morphology containing large nuclei; others were large, more roundly shaped and usually included two or more small dot-like structures within the cytoplasm. Adherent cells were overgrown by thousands of non-adherent small round cells growing heap-like in the center of colonies as shown in FIG. 1A, 2A. Among these cells, we occasionally detected not only a small number of multinucleated osteoclast-like cells, but also highly ramified cells indicative for example of Langerhans and microglia-like cell differentiation. On the surface of some colonies, small satellite colonies (embryoid body-like) consisting of small round cells developed. Parts of these colonies had left the “mother” colony, producing new progeny, which usually displayed a stronger neuronal differentiation capacity than the “mother” colony (FIG. 8).

Performing single cell colony assays we were able to ensure that the observed colonies represented progenies of one single cell. TM-CFU-like growth of definitely single cell-containing wells were further analyzed and showed expression of nestin as well as GFAP and F4/80.

Additionally, we were able to shown that cells of the TM-CFU can be propagated in liquid cultures. In 7-10 days cultures consist of large adherent cells showing the morphology of type II and type III cells and of round cells growing over the adherent cells in form of large clusters. Immunohistochemical staining of the cells showed significant expression of NeuN, the Neuron-specific nuclear protein, in about 20% of cells.

2. Evaluating Growth Factor Requirements

In order to evaluate growth factor requirements for TM-CFU development, cells were cultivated as described in Example 1 and supplemented with either single growth factors or a combination thereof. The most effective cytokines were GM-CSF, and to a lesser extent, IL-3. They also stimulated as single factors TM-CFU growth. SCF, M-CSF and FL alone showed no effect but revealed synergy when combined with GM-CSF and IL-3. LIF with GM-CSF gave also synergistic effects in keeping the cells more undifferentiated. In these cultures colonies appeared about two days earlier and the round cells laying in the center remained larger. LIF combined with M-CSF or SCF showed no effect. NGF or EGF both showed synergistic effects in stimulating neuronal cell differentiation in combination with GM-CSF and IL-3. In cultures supplemented with EGF the neural cells within the surrounding adherent cells developed with more and longer extensions (FIG. 1C). Nevertheless, they were not essential for the neural cell differentiation. In most of the experiments presented here in preselected methylcellulose the combination of IL-3, GM-CSF, SCF and M-CSF was used resulting in an average of 5 TM-CFUs per 5×10³ cells. Detailed analysis however showed that the most favourable growth factor combination in stimulating TM-CFU growth is GM-CSF+IL-3+SCF resulting up to 8 TM-CFU per 5×10³ cells.

3. Phagocytosis of Latex Beads and of Necrotic Liver Tissue Cells

To test the phagocytic activity of colony forming cells (obtained as described in Example 1), phagocytosis was induced by either using FITC-labeled latex-beads or necrotic liver tissue. Phagocytosis was determined by the cells' ability to engulf 1.7 μm-diameter Fluoresbrite fluorescein-coupled carboxylate microspheres (Polysciences Inc.). About 1×10⁵ per μL beads were carefully overlayed on colonies growing in methylcellulose. After 48 h incubation chamber slides were fixed with acetone/methanol. After removal of methylcellulose containing the not phagozytosed beads and washing in phosphate buffered solution (PBS), fluorescence was immediately investigated using an Axionplan 2 fluorescence microscope (Zeiss Inc.). Phagocytic capability of colony forming cells (CFCs) was also investigated using murine necrotic tissue liver cells. Liver was removed, cut in pieces and stored for a few days in serum-free RPMI 1640 medium at 4° C. Small pieces of liver tissue were then placed close to fully developed colonies and chamber slides were incubated for 7-10 days. Cultures were controlled daily. Slides were subsequently fixed with acetone/methanol.

Additionally, mature TM-CFUs in cultures were used to study specific tissue-related conversion (metamorphoses) of CFCs. For this purpose cold-shock-treated liver cells were placed near to the marked TM-CFU and further incubated about 8-10 days. After a period of phagocytosis and ingestion of “injured” cells a change in morphology of CFCs was detected.

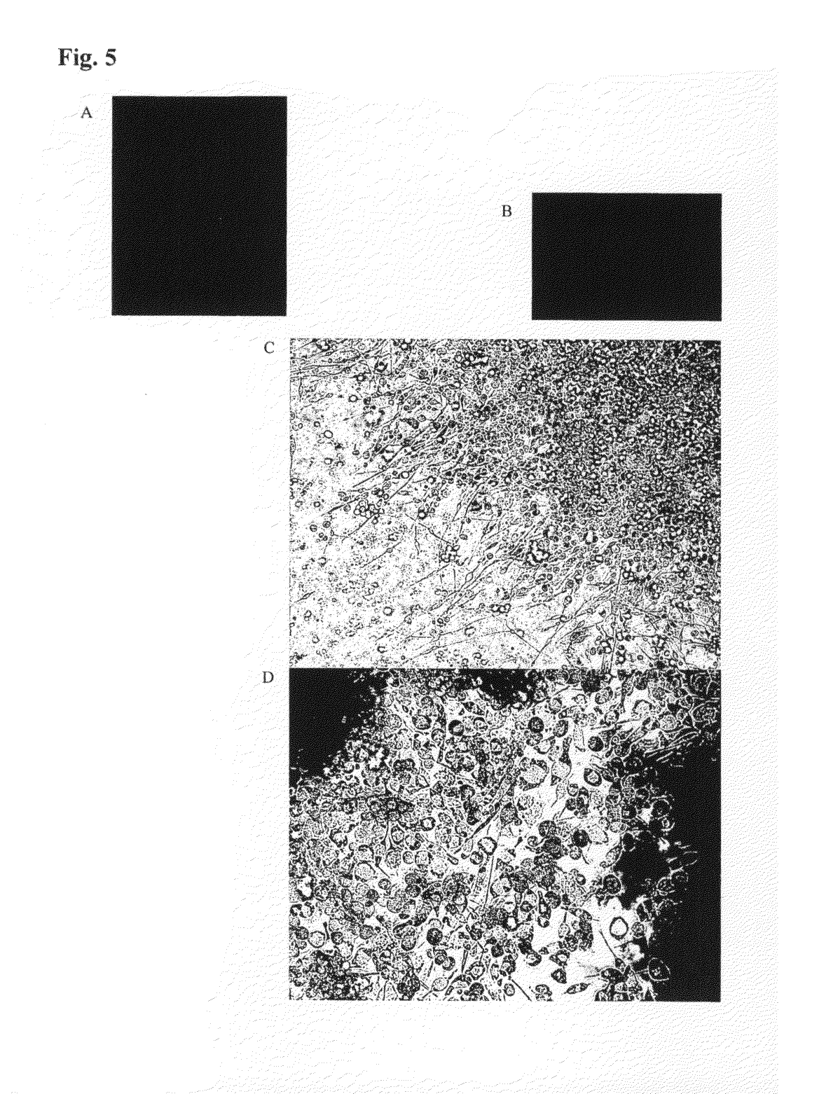

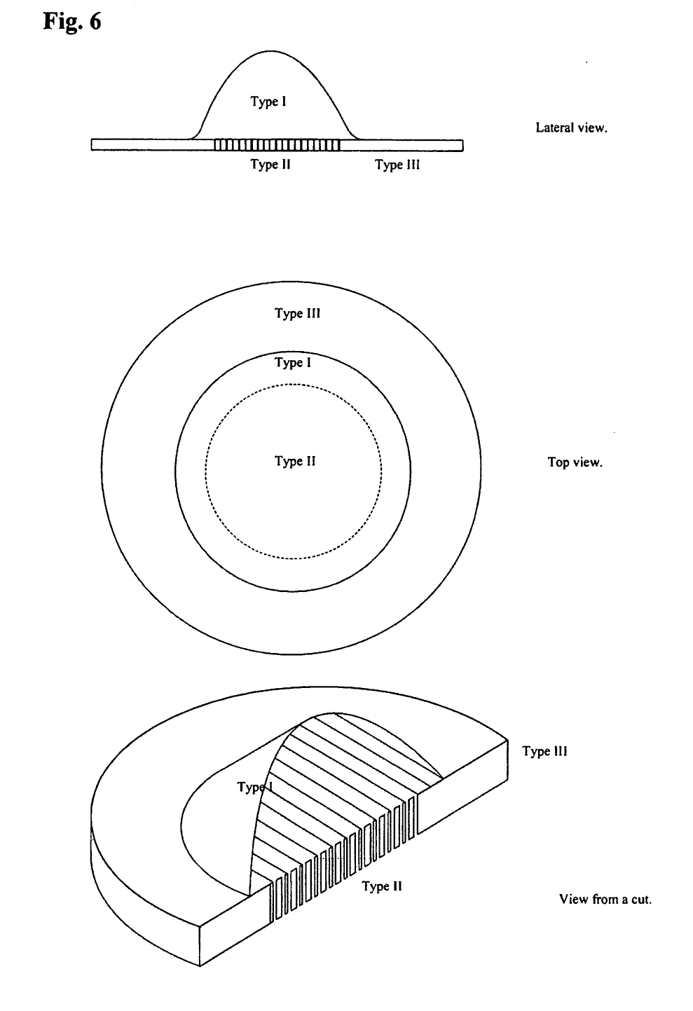

Results: In both cases (using FITC-labeled latex-beads or necrotic liver tissue), clear phagocytic activity of CFCs was observed. FIGS. 5A and 5B show cells with phagocytized FITC-conjugated latex beads. Whereas FITC-labeled cells did not change their morphology, cells incubated with necrotic liver cells underwent distinct morphological alterations (FIGS. 5C and 5D). Near the necrotic tissue, spindle-shaped cells became partly epithelial-like, round or cuboid and their cytoplasm appeared darkly stained resulting from the phagocytized tissue cells (FIG. 5D).

Studying specific tissue-related conversion of CFCs, e.g. by cold-shock, a change in morphology of CFCs could be detected, e.g. the expression of albumin and alpha-fetoprotein. Also a change in the antigen expression pattern of CFCs could be detected. The expression of different MPC phenotypes was investigated on paraformaldehyde-fixed slides using appropriate markers. In the accompanied FIG. 5C and FIG. 5D liver-cell metamorphose is presented. Immunohistochemical studies showed the presence of albumin and alpha-fetoprotein on some cells.

4. Detection of Differentiation Antigens by Immunostaining, FACS-Analysis, Cytochemical Staining and PCR

For immunostaining cells (obtained as described in Example 1) were fixed either with 4% formaldehyde in PBS or acetone/methanol followed by a PBS wash to remove methylcellulose. Briefly, cells were permeabilized in 1% Triton-X-100 for 10 min, then rinsed with PBS and 0.1% Triton-X-100, and subsequently, non-specific binding was blocked by adding 5% goat serum (Dako) for 20 min. For neuron-specific nuclear protein (NeuN) immunostaining, slides were boiled in target retrieval solution, pH 9 (DakoCytomation) for 10 min. Slides were incubated overnight at 4° C. with either the primary antibodies or with normal mouse immunoglobulin or rabbit serum as control. Slides were washed with PBS and incubated for 60 min with the Dako Envision system, goat-anti-rabbit/anti-mouse antibody coupled to alkaline phosphatase. As substrate Fast Red was used (Dako). Finally, all slides were counterstained with Mayer's hematoxylin solution, mounted in Aquatex (Merck) and examined under an Axionplan 2 light microscope (Zeiss Inc.). Images were obtained by a DCC 100 camera (Leica) using Photoshop 5.0 software (Adobe Systems Inc.). All antibodies were applied in 2-4 independent experiments.

For double staining FITC-conjugated mouse-anti-hamster antibody (BD Pharmingen) and Alexa Fluor-conjugated goat-anti-mouse antibody (Molecular Probes) were used as secondary antibodies. For CD11c immunostaining, the primary antibody against CD11c was purchased from BD Bioscience, clone. Slides were counterstained with 4,6 diamidino-2-phenylindole (DAPI, Sigma) and examined under a fluorescence microscope (Leica DMIRE 2). An overlay of fluorescent images showed simultaneous expression of DC-related CD11c and neural cell differentiation-specific III^{β}-tubulin antigens.

The antibodies used in this study are listed in Table 1.

As control cytocentrifuge preparations of isolated bone marrow cells of C57BL/6J were stained with primary antibodies under the same conditions as the test slides of the cultivated CFCs.

For immunofluorescence staining, cells of 5-10 colonies were pooled and labeled with the fluorescein isothiocyanate (FITC)- or phycoerythrin (PE)-conjugated antibodies or FITC- or PE-conjugated istotype controls (dilution 1:50) for 20 min in the dark, at 4° C. Cells were washed two times. Flow cytometric analyses were performed using a FACScan cytometer (Becton-Dickinson). Data were analyzed with Cell Quest software (Becton-Dickinson). All antibodies were applied in 2-4 independent experiments. All antibodies and the isotype-matched PE- and FITC-conjugated antibodies used as negative controls were supplied by BD Biosciences Pharmingen except the mouse-anti-I-A^{b} MHC class II that was obtained from CALTAG Laboratories. Isolated spleen cells were used as positive control.

Monocyte-specific esterase (MSE) staining was performed using α-naphtylacetate as substrate according to the method previously described (Hayhoe, F. G. J. and Quaglino, D. (1994) in Haematological Cytochemistry, Churchill Livingstone, 3d ed., New York). Tartrate resistant acid phosphatase (TRAP) activity was identified by the Leukocyte acid phosphatase kit (Sigma) for 10 min. Slides were counterstained with hematoxylin. Alkaline leukocytophosphatase was investigated using the ALP-kit (Sigma).

For analysis of murine orthologs of matrix metalloproteinase MMP-2, MMP-3 and MMP-9, which increase the migration and recruitment of mononuclear phagocytes in the sites of tissue injury, the expression of MMP-2, MMP-3 and MMP-9 in TM-CFC was performed by one step reverse transcription (RT-PCR) (Qiagen). For this, 200 ng of total RNA of TM-CFCs were used according to the manufacture's instructions by using specific murine MMP-2, MMP-3 and murine MMP-9 primers. In the first step reverse transcription was performed at 50° C. for 30 min. Then the fragments were amplified by PCR with 35 cycles of denaturation (94° C., 30 sec), annealing (64° C., 30 sec), and extension (72° C., 1 min). PCR products were visualized on a 2% agarose gel with ethidium bromide. PCR products were cloned into the pCR TOPO2.1 vector and their specificity was validated by sequencing.

The following primers were used for the RT-PCR:

| | |
|---|---|
| MMP-2 f | 5′gca cac cag gtg aag gat gtg aag 3′ |
| MMP-2 r | 5′c agt taa ggt ggt gca ggt atc tgg 3′ |
| MMP-3 f | 5′gat cca agg aag gca tcc tgt 3′ |
| MMP-3 r | 5′cca tct aca cag ttc aga cac 3′ |
| MMP-9 f | 5′ccg tgc agt gca agt ctc tag aga 3′ |
| MMP-9 r | 5′acc tgg agg aca cag tct gac ctg 3′ |

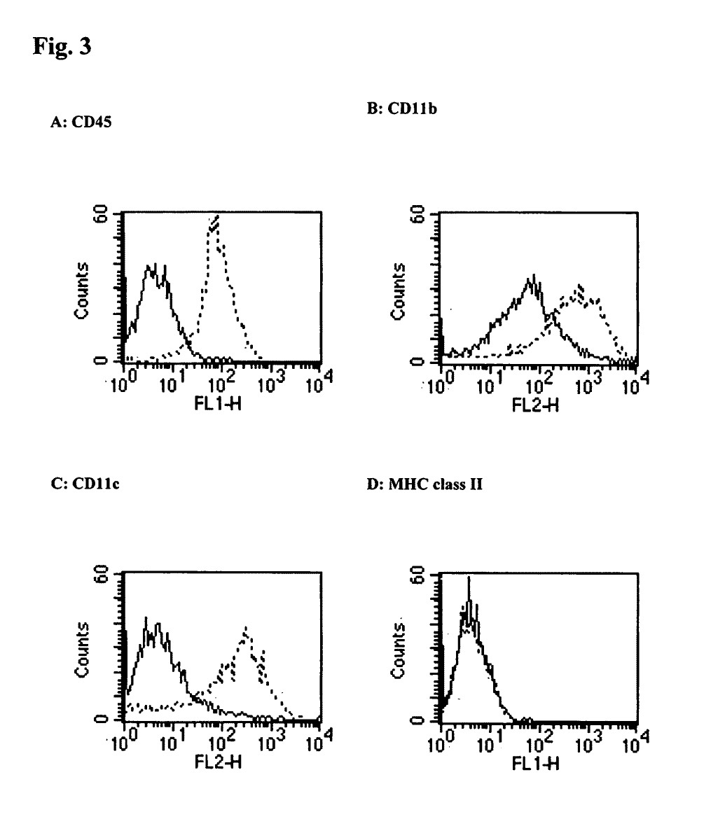

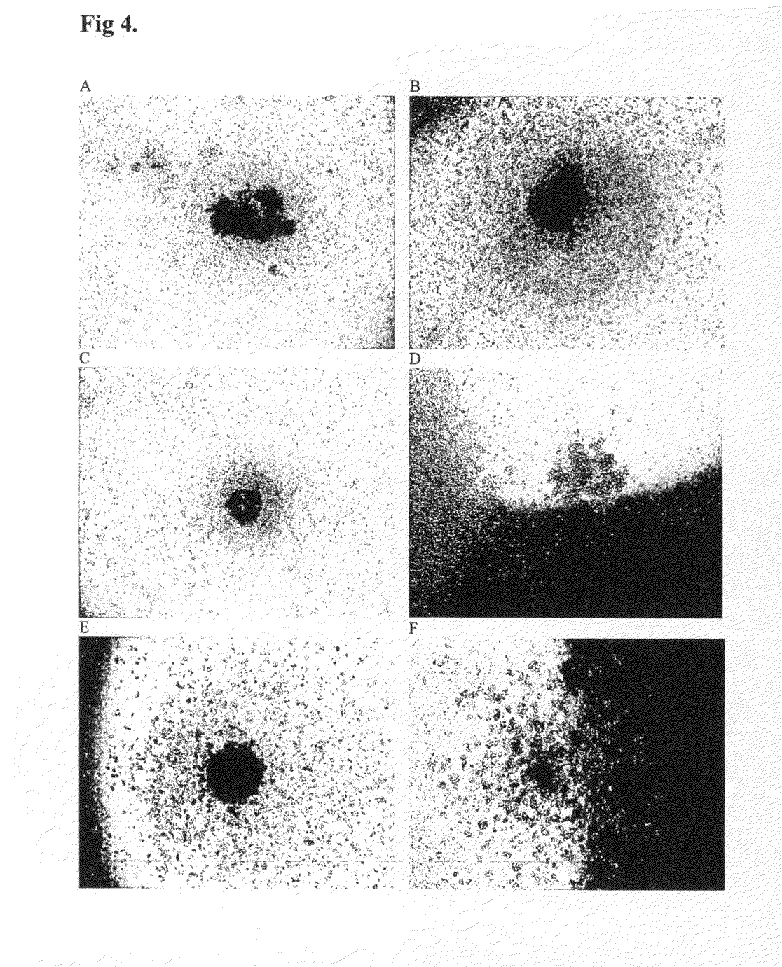

Results: Monocyte/macrophage lineage-specific surface epitopes of CFCs were examined either by FACS-analysis (FIG. 3) or to determine the exact localization of antigen-expressing cells by immunostaining. As controls cytocentrifuge preparations of bone marrow cells were used. Immunostaining of these control preparations with the respective antibodies showed no or only a weak staining on a few cells.

To characterize CFCs, expression of the pan-leukocyte marker CD45 which is exclusively present on cells of the hematopoietic lineage was investigated. As demonstrated by FACS-analysis, about 95% of cells show a positive reaction with the CD45 antibody (FIG. 3A). We analyzed also the expression of the classical monocyte marker CD14. In four independently performed experiment no CD14 expression could be detected (data not shown). Expression of CD11b, the integrin α_{M} chain of MAC-1, was monitored by flow cytometric analysis. Up to 96% of all CFCs were positive for CD11b (FIG. 3B) which was also confirmed by positive immunostaining of cells with CD11b antibody. The presence of antigen-presenting cells in TM-CFU was investigated using antibodies to CD11c, CD80 and CD86. The integrin-α_{κ}chain CD11c, which is expressed in mice mainly on dendritic cells, was found by FACS-analysis on 85% of cells (FIG. 3C). Expression of CD80 and CD86 found on mature antigen-presenting cells was detectable only on 13-17% of the cells. The expression of the class II MHC molecule was also analysed. In four independent experiments no expression of I-Ab encoded MHC class II antigen was detected (FIG. 3D), not even after INF-γ stimulation (data not shown).

The presence of heterogenous MPC phenotypes was examined by extended analyses of early (CD115, CD205 and S-100) and late (F4/80, CD163, MAC-3 and Iba-1) mononuclear phagocytic markers. Immunostaining for the M-CSF receptor, c-fms (CD115), and for the multilectin domain molecule DEC205, as well as for S-100 revealed positive signals indicating the presence of a rather immature MPC population. Expression of stem cell factor receptor (c-kit) using CD117 antibody resulted in a clear signal which is in accordance with in vitro culture studies proving SCF as an important synergistic factor to GM-CSF and IL-3. In line with these findings expression of the CD34 stem cell marker could be observed. Using the antibody against the stem cell marker CD90 resulted in a strong signal on the TM-CFCs.

F4/80 usually detects a well-defined cell population of MPS, including dendritic, Langerhans and microglial cells. F4/80-positive cells were distributed throughout colonies. Anti-CD163 detecting the ED2 surface glycoprotein expressed by macrophages, as well as anti-MAC-3 resulted in a clear positive staining.

The scavenger receptor CD91/low-density lipoprotein receptor-related protein, which is among others required to efficiently internalize antigens by CD11c⁺ dendritic cells showed positive immunostaining, especially on cells in the center of colonies.

The monocytic/macrophage origin of CFCs was also investigated by cytochemical stainings. TM-CFU composing cells showed independent of their morphological diversity a strong staining with MSE, the monocyte-specific esterase. Furthermore, most of the CFCs displayed also significant tartrate resistant acid phosphatase, TRAP-activity, an enzyme which is known to be highly expressed in osteoclasts and in a subset of tissue macrophages and dendritic cells. It is noteworthy, that many cells with neural cell-like morphology showed also positive MSE- and TRAP-staining. However no alkaline leukocytophosphatase could be detected. The phagocytic activity of CFCs was demonstrated by FITC-labelled latex-beads and necrotic liver tissue cells.

Proteins that control cellular adhesion and motility play a crucial role in all processes of MPCs such as migration, phagocytosis and tissue regeneration. The expression of cytoskeleton-associated proteins and extracellular matrix (ECM) components was shown. The antibody to the Ca⁺⁺-dependent adhesion molecule E-cadherin, which mediates cell-cell interactions critical for morphogenesis, resulted in a very strong staining in the center of colonies. Similarly strong staining was observed using an antibody to the cytoskeleton-related protein actin. Vimentin, a member of intermediate filaments, mainly expressed on cells of mesenchymal origin and to a lesser extent on glial cells, was detected by a rather inhomogeneous reaction. The expression of ECM-related antigens was also investigated. Positive, but inhomogeneous staining was found for collagen type II. Collagen type IV expression of basement membranes was observed on diverse CFCs.

Strong staining on all CFCs was observed by using an antibody to fibronectin which mediates cell-cell adhesion processes. A comparable strong positive reaction was detected with an antibody to the widely distributed ECM-protein laminin. The morphogenesis-related MMPs, like MMP-1, MMP-2, MMP-9 as well as MMP-3, as well as one of their inhibitors TIMP-2, all responsible for the degradation as well as the re-modeling of ECM components were found to be expressed in many of the TM-CFUs. MMP-1 antigen was only detected on a few cells. Specifically the MMP-9 expression was found to be high by RT-PCR.

To examine the ability of certain CFCs to mature along the neuronal cell lineage, expression of several neuronal markers was studied by immunostaining. Clear neuronal cell differentiation was detected by antibodies to nestin, NSE, NF-200, class III^{β}-tubulin, MAP-2a,b, NeuN and synaptophysin as well as differentiation of astroglial cells using anti-GFAP. Simultaneous expression of the MPC-related marker CD11c and the neural cell marker class III^{β}-tubulin were observed on several CFCs.

In conclusion, these results show 1) the development of the MPS from common cells, 2) the anchoring of neural cell differentiation within the MPS development, and 3) the generation of mesenchymal and neuroectodermal differentiation in one and the same progeny. We showed that cells making up the TM-CFU express, among others, a variety of monocytic/macrophage differentiation antigens and display a remarkably diverse morphology. The strong overlapping antigen expression pattern of TM-CFCs revealed a continuum of MPCs instead of clear definable phenotypes.

The strong expression of morphogenesis-related proteins such as E-cadherin and actin as well as diverse ECM components such as fibronectin, laminin, collagen and MMPs implies that TM-CFCs play a role in regenerative processes.

Strong MMP-9 expression was found in TM-CFCs by RT-PCR and less, but still high MMP-2 expression was detected in TM-CFCs.

The results of marker expression are summarized in Table 1:

TABLE 1
Marker expression in TM-CFU

| Primary antibody | | Control | | |
|---|---|---|---|---|
| (Clone) | Specificity | Antibody | Company | Expression |
| CD 45 (IBL-5/25) | Panleucocyte marker | Rat IgG | Neo Markers | +++++ |
| CD45-FITC (30-F11) | Panleucocyte marker | Rat IgG2b- | BD Biosciences | >90% |
| | | FITC | Pharmingen | |
| CD14-PE (rmC5-3) | Receptor of | Rat IgG1-PE | BD Biosciences | No |
| | lipoploysaccharid complex | | Pharmingen | expression |
| mouse-anti-I-A^{b} MHC | MHC class II alloantigen | Mouse IgM- | CALTAG | No |
| lass II-FITC (25-5- | | FITC | Laboratories | expression |
| 16S). | | | | |
| CD90 (OX-7) | Thy-1.1, stem cell marker | Mouse-IgG1 | BD Biosciences | +++(+) |
| | | | Pharmingen | |
| F4/80 (Cl:A3-1) | 160 kDa glycoprotein of | Rat-IgG2b | Serotec | +++ |
| | macrophages | | | |
| CD11b-PE (M1/70) | MAC-1, Integrin monocyte/ | Rat IgG2b- | BD Biosciences | >80% |
| | macrophage marker | PE | Pharmingen | |
| CD11c-PE (HL3) | Integrin dendritic cells/low | Hamster IgG- | BD Biosciences | >80% |
| | density cells marker | PE | Pharmingen | |
| CD205 (DEC205, purified | Multilectin receptor, dendritic | Rat-IgG2a | Dr. K. Mahnke, | +++ |
| from hybriddoma | cell marker | | (DKFZ Heidelberg, | |
| cell line NLDC125) | | | Germany). | |
| CD80-FITC (16-10A1) | B7-1, costimulatory | Hamster IgG- | BD Biosciences | 10% |
| | molecule | FITC | Pharmingen | |
| CD86-PE (GL1) | B7-2, costimulatory | Rat IgG2a- | BD Biosciences | 10% |
| | molecule | PE | Pharmingen | |
| MAC-3 (M3/84) | Macrophage differentiation | Rat-IgG1 | BD Biosciences | +++ |
| | antigen | | Pharmingen | |
| CD91 (5A6) | Low-density lipoprotein | Mouse-IgG1 | Progen | +++ |
| | receptor-related protein | | | |
| CD163 (ED2) | ED2 glycoprotein of | Mouse | Serotec | +++ |
| | macrophages | | | |
| Iba 1 | Microglial differentiation | Rabbit-serum | Wako Chemicals, | ++++ |
| | | | Japan | |
| CD115 (AFS 98) | c-fms, Colony stimulating | Rat-IgG2a | BD Biosciences | ++ |
| | factor 1 receptor | | Pharmingen | |
| CD34 (MEC14.7) | Glycoprotein of lympho- | Rat-IgG2a | HyCult | ++ |
| | hematopoietic progenitor | | Biotechnology | |
| | cells | | | |
| CD117 (180627) | Stem cell factor receptor | Rat IgG2a | BD Biosciences | ++(+) |
| | | | Pharmingen | |
| CD135 (4G8) | FMS-like tyrosine kinase 3 | Mouse IgG1 | BD Biosciences | ++ |
| | (flt3) | | Pharmingen | |
| Vimentin (VIM 3B4) | Intermediate filament protein | Mouse-IgG2a | Boehringer- | ++ |
| | | | Mannheim | |
| E-cadherin (5H9) | Ca⁺⁺-dependent adhesion | Mouse-IgG2a | Progen | ++++ |
| | molecule | | | |
| Fibronectin (FBN11) | Extracellular matrix dimeric | Mouse-IgG1 | NeoMarkers | +++++ |
| | protein | | | |
| Collagen type II (2B1.5) | Cartilage matrix protein | Mouse-IgG2a | NeoMarkers | +++ |
| Laminin (909256) | Extracellular matrix protein | Rabbit-serum | Becton-Dickinson | +++++ |
| MMP1 (41-1E5) | Matrix-metalloproteinase | Mouse-IgG2a | Oncogene Res. | + |
| MMP3 (55-2A4) | Matrix-metalloproteinase | Mouse-IgG1 | Oncogene Res. | + |
| TIMP2 (67-4H11) | Tissue inhibitor of matrix- | Mouse-IgG1 | Oncogene Res. | + |
| | metalloproteinases, tissue | | | |
| | remodeling | | | |
| S100 | Ca-binding protein | Rabbit serum | DAKO | ++++ |
| Nestin (rat-401) | Major intermediate filament | Mouse-IgG1 | Chemicon Int. | +++++ |
| | of nervous progenitor cells | | | |
| Neuron-specific | Glycotic isoenzyme of | Rabbit-serum | Chemicon Int. | ++++ |
| enolase (NSE), | enolase gamma-gamma | | | |
| polyclonal | dimmer of neurons | | | |
| GFAP (6F2) | 52 kDa intermediate filament | Mouse-IgG1 | DAKO | +++ |
| | protein, astrocyte marker | | | |
| Neuron-specific nuclear | Neuronal nuclei | Mouse-IgG1 | Chemicon Int. | ++ (+) |
| protein, NeuN, (A60) | | | | |
| Class III^{β}-tubulin (TU- | Class III- [symbol] -isoform of tubulin | Mouse-IgG1 | Chemicon Int. | ++ |
| 20) | | | | |
| Synaptophysin (SY38) | Synaptic vesicle regulatory | Mouse-IgG1 | DAKO | +++ |
| | protein | | | |
| Neurofilament-200, NF- | Neural specific antigen | Mouse-IgG1 | Sigma | +++ |
| 200 (N52) | | | | |
| Microtubule-associated | Microtubulin-associated | Mouse-IgG1 | NeoMarkers | +++ |
| protein-2a,b (MAP- | protein | | | |
| 2a,b) (AP20) | | | | |
| CD 31 (MEC 13.3) | Endothelial cell | Rat IgG2a | BD Biosciences | ++ |
| | differentiation | | Pharmingen | |
| Pan Cytokeratin (80) | Epithelial cell differentiation | Mouse IgG1 | Acris | ++(+) |
| Albumin (188835) | Liver cell differentiation | Mouse IgG2a | R&D Systems | + |
| Alpha Fetoprotein | Liver cell differentiation | Mouse IgG1 | R&D Systems | + |
| (189502) | | | | |
| Actin (1A4) | Actin filament protein | Mouse-IgG2a | Beckman-Coulter | +(+) |
| BMP 4 (N-16) | Bone morphogenetic protein | Goat-serum | Santa Cruz | + |
| BMP 5 (N-19) | Bone morphogenetic protein | Goat-serum | Santa Cruz | + |
| Glucagon, | Hormone: glucagon | Rabbit-serum | Zymed Lab. | (+) |
| polyclonal | | | | |
| Insulin, | Hormone: insulin | Guinea-pig | Dako | (+) |
| polyclonal | | serum | | |
| Vasoactive intestinal | Peptide hormone | Rabbit-serum | BioGenex | ++ |
| peptide, | | | | |
| polyclonal | | | | |

| Marker | | PCR primer | Expression |
|---|---|---|---|
| MMP-9 | Matrix-metalloproteinase | mouse-specific | +++++ |
| | | primer | |
| MMP-2 | Matrix-metalloproteinase | mouse-specific | +++ |
| | | primer | |
| MMP-3 | Matrix-metalloproteinase | mouse-specific | + |
| | | primer | |

(very strong expression. +++++, strong expression. ++++, middle expression. +++, weak expression: ++, very weak expression: +, each referring to ratio of positive cells; the numbers define the ratio of positive cells) [symbol]
indicates data missing or illegible when filed

The co-differentiation of MPS and neural cells was surprising, being a “spontaneous” event in the progeny which occurred even without specific neural cell growth promoting cytokines. These results indicate strongly that the neural cell precursors of TM-CFU are the colony-forming microglia cells. The biological significance of co-differentiation of MPCs and neuronal cells should not be underestimated. Both, the MPS and the neuroendocrine system are responsible for maintaining the biological equilibrium in the adult organism. This includes also tissue cell regeneration. Nevertheless, in TM-CFUs also further examples for various differentiation capacities were detected. Noteworthy are the epithelial differentiation (cytokeratin expression), the endothelial differentiation (CD31 expression) and the chondrocyte differentiation (collagen 2 expression) on a few TM-CFCs without specific stimulation. Furthermore, neuroendocrine differentiation capacities of TM-CFCs were observed (insulin, glucagon and vasoactive intestinal peptide expression).

5. TM-CFU Derived from Human Samples

Samples of human cord blood and human bone marrow were obtained and used for the preparation of TM-CFU. Furthermore, TM-CFUs were produced from human blood after apheresis following mobilisation of progenitor cells with GCF (granulocyte colony stimulating factor).

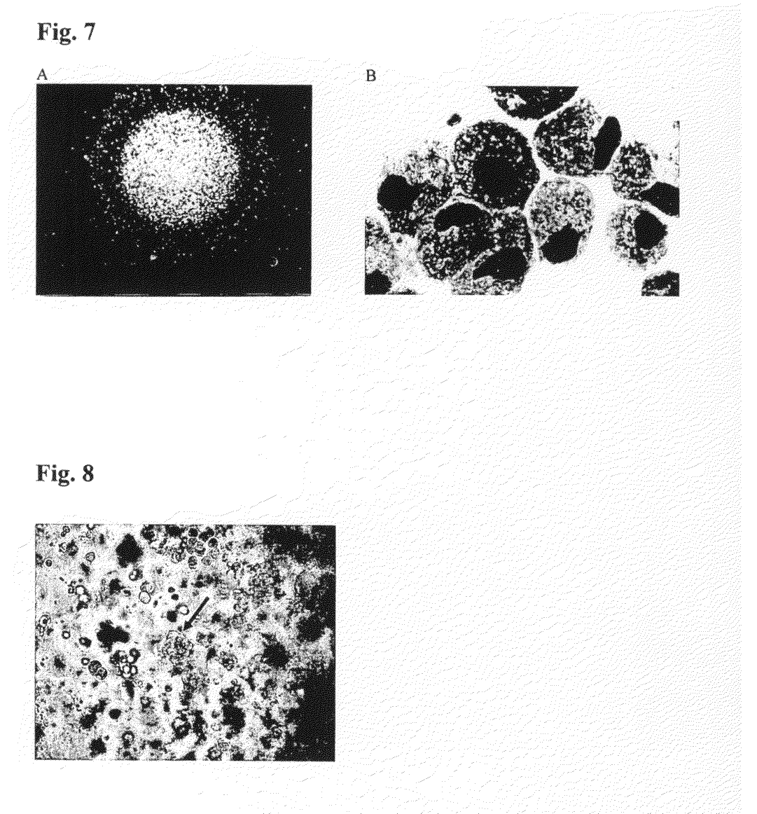

In the course of the haematological special diagnostic in our laboratory human bone marrow cells, G-CSF mobilized peripheral blood cells and cord blood cells were investigated. Cells were isolated using Ficoll gradient centrifugation. Mononuclear cells of the interphase were collected and washed in buffer. The number of viable cells was estimated using acridin orange staining. 1×10⁴/ml semi-solid medium (agar or methylcellulose) were cultivated in the presence of growth factors GM-CSF, IL-3 50 ng/ml each and SCF 20 ng/ml. After two to three weeks of incubation at 37° C. under a fully humidified atmosphere and 6.5% C0₂ colony numbers and sizes were evaluated using dissection microscope. The number of TM-CFUs was highly dependent on the biological situation of patients. In general 0-10 TM-CFU per 1×10⁴ cells could be detected. FIG. 7A shows a human TM-CFU of a healthy donor after G-CSF mobilization growing in agar culture. Note the surrounding halo-like growing type III cells in the colony. FIG. 7B shows the isolated cells of this colony after Giemsa-staining clearly demonstrating the round cells bearing eccentric located nuclei.

Similar investigations were done with cord blood cells. Cells were isolated using Ficoll gradient centrifugation. Mononuclear cells of the interphase were collected and washed in buffer. The number of viable cells was estimated using acridin orange staining. 1×10⁴ cells/ml semi-solid methylcellulose medium were cultivated in the presence of growth factors GM-CSF and IL-3 at 25 ng/ml each and SCF at 20 ng/ml. After approx. two weeks of incubation at 37° C. under a fully humidified atmosphere and 6.5% C0₂ colony number and size were evaluated using dissection microscope. In the cord blood investigated we detected only 2 TM-CFU/1×10⁴ cells. Staining of these TM-CFCs with anti-GFAP antibody showed positive reactions.

Results: In all probes investigated TM-CFUs appeared, but the yield of TM-CFUs depends on the given biological situation of the “patient”. The in the human system observed TM-CFUs showed identical morphology with the murine TM-CFUs. As far as tested we observed e.g. expression of identical markers (NeuN, GFAP, F4/80) in the human TM-CFCs compared to murine TM-CFCs.

6. In Vivo Regeneration

The regeneration capacity of TM-CFCs in vivo was investigated using TM-CFUs from bone marrow of male transgenic mice overexpressing Green fluorescent protein (GFP) under the promoter of actin (strain C57BL/6-Tg(ACTB-EGFP)10sb/J, Jackson Laboratories). Mature TM-CFUs were prepared as detailed in Example 1 and were collected from methylcellulose under sterile conditions in PBS. 10 TM-CFUs per mouse were prepared for transplantation. Cells were transplanted intraperitoneally into anestesized wild type female C57BL/6J mice after opening of the abdominal cavity and setting of pancreatic injury. At the end of the operation the peritoneum and the skin were closed by sutures. After two to eight weeks sections of mice were performed and bone marrow, spleen, pancreas, scar, skin, peritoneum, lung, liver, and brain were excised. Analysis of GFP-expressing cells in different organs were performed either directly using a fluorescence microscope or with a light microscope after in-situ hybridization with a probe for murine y-chromosome (Cambio) or after immunhistochemistry using a specific goat anti-mouse GFP antibody (Abcam).

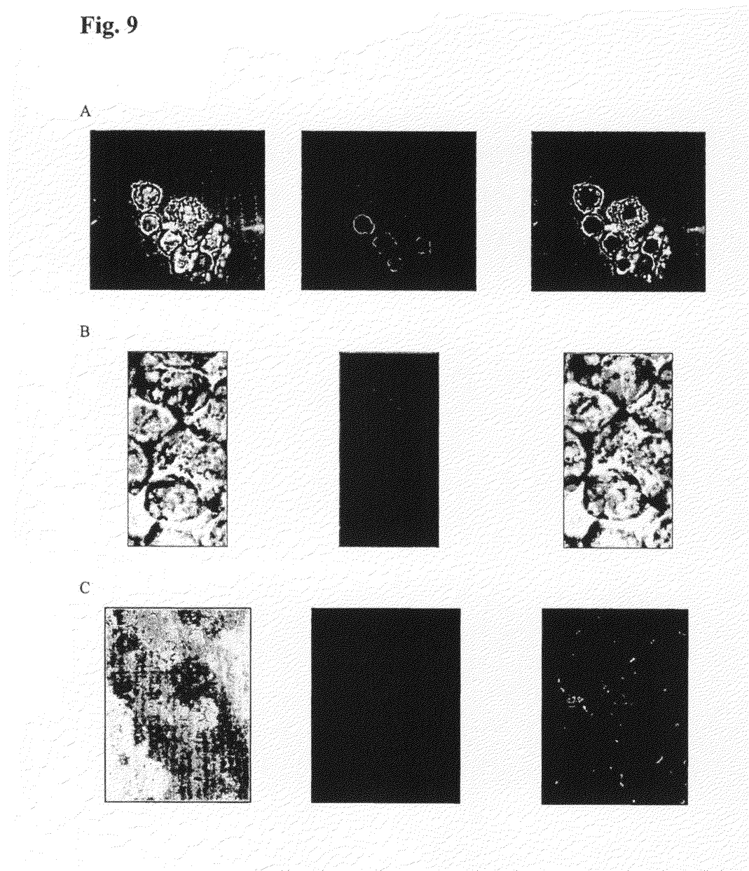

Results: GFP-expressing cells were observed in bone marrow, spleen, skin, lung and pancreas demonstrating that the TM-CFCs are able to migrate into different organs (FIG. 9). The TM-CFUs were able to bed into the injured pancreas starting the regeneration process.

7. In Vivo Migration

The capacity of TM-CFCs in vivo to migrate to the site of injury was investigated using TM-CFUs from bone marrow of male mice. Mature TM-CFUs prepared as detailed in Example 1 were collected from methylcellulose under sterile conditions in PBS. 10-15 TM-CFUs per mouse were pooled for transplantation. Cells were labeled with a fluorescent infrared emitting dye, NHS ester CY5.5 according to the manufactures instructions (G.E. Healthcare), and transplanted intraperitoneally into anestesized nude mice (NMRI-Fox nu/nu; Harlan-Winkelmann) after setting a very small intradermal incision at the left shoulder followed by a cryo-injury using a small instrument of copper filled with nitrogen. In order to detect CY5.5-labeled TM-CFCs in mice at the site of injury over time images of mice were acquired using a time domain small fluorescence imager, the eXplore Optix system (General Electrics, Global Research). This system allows observation of fluorescent signals from larger tissue depth and the determination of fluorescence intensity as well as identification of fluorescence lifetime characteristic for each fluorescence dye. The device uses a pulse laser diode having a wavelength in the near-infrared region of 670 to 700 nm. Scans of mice monitoring the area of interest (injury) were performed at distinct time points with the same parameters. The presence of CY5.5 labeled cells at the site of injury was verified by histology analyses at the end of the experiment. 3 weeks after implantation of cells mice were sacrificed and scar tissue was excised. Frozen sections were performed and analyzed by fluorescence microscopy (Microscope Axiowert200M, camera Yamatsu ORCA ER C4742.18). Cells showing near infrared fluorescence were detected at the skin lesion already regenerated.

Results: Fluorescent signals characteristic for Cy5.5 labeled TM-CFCs were detected with the time domain small fluorescence imager (eXplore Optix) at the site of the injured skin of the mouse over time in vivo demonstrating that the TM-CFCs are able to migrate into the cryo-lesion of the skin. CY5.5 labeled cells are already detectable at the site of injury after 24 hours and can be monitored up to 120 hours by eXplore Optix.

8. Long-Term Experiment

The capacity of TM-CFCs in vivo to develop tumors was investigated using TM-CFUs from bone marrow of mice. Mature TM-CFUs prepared as detailed in Example 1 were collected from methylcellulose under sterile conditions in PBS. 10-15 TM-CFUs per mouse were pooled for subcutaneous implantation into SCID mice (n=2). After ten months no tumor growth was visible at site of implantation.

1-36. (canceled)

37. A method of preparing a tissue-maintaining colony-forming unit (TM-CFU) consisting of CD14 negative cells, the method comprising the steps of:
(a) cultivating, in the presence of Granulocyte/Macrophage Colony-Stimulating Factor (GM-CSF) and/or Interleukin-3 (IL-3), cells from bone marrow, blood, umbilical cord or skin; and
(b) isolating said TM-CFU formed in step (a),
wherein the TM-CFU is further defined by the presence of
(i) a majority of a first group of cells, wherein the cells are round with an eccentric nucleus and grow non-adherently, hillock-like in the center of the TM-CFU,
a second group of cells, wherein the second group of cells includes cells with extensions and cells having cuboid- or triangle-shaped morphology and wherein the cells of the second group are adherent and larger than the cells of the first group and grow underneath the first group of cells,
a third group of cells, wherein the third group of cells includes cells with extensions and spindle-shaped cells and wherein the cells are adherent, have variable morphology and grow around the second group of cells, and
optionally satellite colonies developed in the center of the TM-CFU and showing embryoid body-like morphology;
and/or
(ii) the CD45 antigen.

38. The method of claim 37, wherein at least 80 of the total number of cells of the TM-CFU are CD45 positive.

39. The method of claim 37, wherein the number of cells of the first group of cells amounts to at least 60% of the total number of cells of the TM-CFU, wherein the number of cells of the second group of cells amounts to approximately 1% to 30 of the total number of cells of the TM-CFU and wherein the number of cells of the third group of cells amounts to approximately 1% to 30 of the total number of cells of the TM-CFU.

40. The method of claim 37, wherein the number of the first group of cells amounts to approximately 80% to 94% of the total number of cells of the TM-CFU, wherein the number of the second group of cells amounts to approximately 3% to 10% of the total number of cells of the TM-CFU and/or wherein the number of the third group of cells amounts to approximately 3% to 10% of the total number of cells of the TM-CFU.

41. The method of claim 37, wherein the TM-CFU has a diameter of approximately at least 2 mm.

42. The method of claim 37, wherein the cultivation period of step (a) is carried out over approximately 6 to approximately 16 days.

43. The method of claim 37, wherein the cells of the first group of cells have an average diameter of approximately 5 to 50 μm and wherein the cell bodies of the second group of cells have an average diameter of at least approximately 25 μm.

44. The method of claim 37, wherein in step (a) the cells are cultivated in a single cell format.

45. The method of claim 44, wherein the TM-CFU is derived from a single cell.

46. The method of claim 37, wherein at least one factor selected from the group consisting of Stem Cell Factor (SCF), Ftl-3 ligand (FL) and Macrophage Colony Stimulatory Factor (M-CSF) is additionally present in step (a).

47. The method of claim 37, wherein in step (a) Leukemia Inhibitory Factor (LIF) is additionally present.

48. The method of claim 37, wherein the TM-CFU contains Glial Fibrillary Acidic Protein (GFAP) positive cells and Neuronal Nuclei (NeuN) positive cells.

49. The method of claim 37, wherein the cells of the TM-CFU are alkaline phosphatase negative.

50. The method of claim 37, wherein the cells of the TM-CFU are HLA-DR II negative.

51. The method of claim 37, wherein the cells of the TM-CFU show phagocytic activity.

52. The method of claim 37, wherein the cells of the TM-CFU are capable of spontaneously differentiating into cells of the mononuclear phagocytic system and neural cells without adding a differentiation-inducing agent to the medium used for cultivation.

53. A TM-CFU consisting of CD14 negative cells, wherein the TM-CFU is further defined by the presence of
(i) a majority of a first group of cells, wherein the cells are round with an eccentric nucleus and grow non-adherently, hillock-like in the center of the TM-CFU,
a second group of cells, wherein the second group of cells includes cells with extensions and cells having cuboid- or triangle-shaped morphology and wherein the cells of the second group are adherent and larger than the cells of the first group and grow underneath the first group of cells,
a third group of cells, wherein the third group of cells includes cells with extensions and spindle-shaped cells and wherein the cells are adherent, have variable morphology and grow around the second group of cells, and
optionally satellite colonies developed in the center of the TM-CFU and showing embryoid body-like morphology;
and/or
(ii) the CD45 antigen.

54. The TM-CFU of claim 53, wherein the TM-CFU is derived from a mammal.

55. Pharmaceutical composition comprising the TM-CFU according to claim 53 and optionally excipients and/or auxiliaries.

56. A method of treating a subject being in need of maintaining, generating or regenerating a tissue, comprising administering to the subject an effective amount of cells from the TM-CFU according to claim 53.

57. The method of claim 56, wherein the tissue is an endodermic, mesodermic and/or an ectodermic tissue.

58. The method of claim 56, wherein the tissue is located in an organ selected from the group consisting of the skin, the eye, the nose, the ear, the brain, the spinal cord, a nerve, the trachea, the lungs, the mouth, the esophagus, the stomach, the liver, the small intestines, the large intestines, the kidney, the ureter, the bladder, the urethra, a gland such as hypothalamus, pituitary, thyroid, pancreas and adrenal glands, the ovary, the oviduct, the uterus, the vagina, a mammary gland, the testes, the penis, a lymph nodes, a vessel, the heart, a blood vessel, a skeletal muscle, a smooth muscle, a bone, cartilage, a tendon and a ligament.

59. The method of claim 56, wherein the subject is suffering from a pathological condition or a disease.

60. The method of claim 59, wherein the condition or disease is selected from the group consisting of from cancer, an autoimmune disease, a neurodegenerative disease, a respiratory disease, a vascular disease, diabetes mellitus, Alzheimer's disease, Lewy body dementia, Parkinson's disease, a trauma, burn, head trauma, spinal cord injury, stroke, myocardial infarction, arthrosis, Huntington's disease, Tourette's syndrome, multiple sclerosis, amyotrophic lateral sclerosis, Addison's disease, pituitary insufficiency, liver failure, inflammatory arthropathy, neuropathic pain, blindness, hearing loss, arthritis, a bacterial infection, a viral infection, a sexually transmitted disease and a damage of the skin, the eye, the nose, the ear, the brain, the spinal cord a nerve, the trachea, the lungs, the mouth, the esophagus, the stomach, the liver, the small intestines, the large intestines, the kidney, the ureter, the bladder, the urethra, a gland such as hypothalamus, pituitary, thyroid, pancreas and adrenal glands, the ovary, the oviduct, the uterus, the vagina, a mammary gland, the testes, the penis, a lymph nodes, a vessel, the heart, a blood vessel, a skeletal muscle, a smooth muscle, a bone, cartilage, a tendon or a ligament.

61. Method of determining the effect of at least one stimulus on the TM-CFU according to claim 53 or a cellular subpopulation thereof:
(a) exposing the TM-CFU to the at least one stimulus; and
(b) determining the effect of the at least one stimulus on the TM-CFU or a cellular subpopulation thereof.


SEQUENCE LISTING (6 sequences)

SEQ ID NO: 1 - DNA, 24 bp, Artificial Sequence
  Description of Artificial Sequence Synthetic primer
gcacaccaggtgaaggatgtgaag

SEQ ID NO: 2 - DNA, 25 bp, Artificial Sequence
  Description of Artificial Sequence Synthetic primer
cagttaaggtggtgcaggtatctgg

SEQ ID NO: 3 - DNA, 21 bp, Artificial Sequence
  Description of Artificial Sequence Synthetic primer
gatccaaggaaggcatcctgt

SEQ ID NO: 4 - DNA, 21 bp, Artificial Sequence
  Description of Artificial Sequence Synthetic primer
ccatctacacagttcagacac

SEQ ID NO: 5 - DNA, 24 bp, Artificial Sequence
  Description of Artificial Sequence Synthetic primer
ccgtgcagtgcaagtctctagaga

SEQ ID NO: 6 - DNA, 24 bp, Artificial Sequence
  Description of Artificial Sequence Synthetic primer
acctggaggacacagtctgacctg